(19) United States
(12) Patent Application Publication
(10) Pub. No.: US 2014/0037700 A1
(43) Pub. Date: Feb. 6, 2014

(54) Composition and Method for Enhancing an Immune Response

(71) Applicant: Jason Fisher, New York, NY (US)

(72) Inventor: Jason Fisher, New York, NY (US)

(73) Assignee: MICO BIO, INC., New York, NY (US)

(21) Appl. No.: 14/112,762

(22) PCT Filed: Apr. 19, 2012
(86) PCT No.: PCT/US12/34221

§ 371 Date: Oct. 18, 2013

Related U.S. Application Data
(60) Provisional application No. 61/477,284, filed on Apr. 20, 2011.
(60) Provisional application No. 61/477,306, filed on Apr. 20, 2011.
(60) Provisional application No. 61/477,353, filed on Apr. 20, 2011.
(60) Provisional application No. 61/477,385, filed on Apr. 20, 2011.
(60) Provisional application No. 61/477,369, filed on Apr. 20, 2011.
(60) Provisional application No. 61/488,530, filed on May 20, 2011.

(51) Int. Cl. A61K 39/02; A61K 31/353; A61K 31/59; A61K 31/203; A61K 39/09; A61K 39/04

(52) U.S. Cl. CPC A61K 39/098; A61K 39/02; A61K 39/092; A61K 39/04; A61K 31/59; A61K 31/203; A61K 31/353
USPC 424/400; 424/234.1; 424/237.1; 424/248.1; 424/252.1; 435/252.1; 435/253.1

(57) ABSTRACT

Disclosed are methods and compositions for treating or preventing microbial infections.

### RELATED APPLICATIONS

This application claims priority to U.S. Ser. No. 61/477,353, filed Apr. 20, 2011; U.S. Ser. No. 6161/477,369, filed Apr. 20, 2011; U.S. Ser. No. 61/477,369, filed Apr. 20, 2011; U.S. Ser. No. 61/477,385, filed Apr. 20, 2011; U.S. Ser. No. 61/477,284, filed Apr. 20, 2011; U.S. Ser. No. 61/477,306, filed Apr. 20, 2011; and U.S. Ser. No. 61/488,530, filed May 20, 2011. The contents of these applications are incorporated herein by reference in their entireties.

### FIELD OF THE INVENTION

The invention relates to method for enhancing an immune response and more particularly to a vaccine using inactivated Mycobacterium spp.

### BACKGROUND OF THE INVENTION

Mycobacterium tuberculosis (M. tb) infects one third of the world's human population^{[1]}. The common tuberculosis (TB) vaccine known as the Bacillus Calmete Guerrin (BCG) vaccine is given to neonates in developing countries. While this vaccine protects against meningeal and disseminated TB in children, it fails to adequately protect the establishment of latent TB or reactivation of pulmonary disease in adult life^{[2]}. Moreover, BCG effectiveness is reported to decline over a period of 10-15 years^{[3]}. The most common type of tuberculosis disease is pulmonary, and transmission occurs via aerosol droplets expressed during coughing. Thus, despite the high prevalence of BCG vaccination, the disease burden has not decreased. There is now evidence that M.tb microbacterial lineages may have adapted to mutations in antigens common to both M.tb and BCG^{[4,5]}. Moreover, recent studies suggest that BCG delivered parenterally may fail to induce T-cell immune responses in the lung mucosa, which may be critical for protection against pulmonary disease^{[6,7]}. For at least these reasons, a new vaccine is imperative to decrease the prevalence of TB throughout the world.

### SUMMARY OF THE INVENTION

The invention is based on the discovery of new ways of preparing compositions to increase a desired immune response.

In one aspect, the invention provides a vaccine using a population of irradiated M.tb including a high percentage of cells in a predetermined biological state. A state of M.tb can mean, e.g., cells in metabolic states arising from nutritional deprivation, extreme temperatures, iron depletion, aerobic growth, anaerobic growth, oxidative stress, or a combination of two or more of these states. In some embodiments, more than 90%, 95%, 98%, 99% or 99.9% of the M.tb cells are in the predetermined state.

The vaccine can be used along with a number of other vaccination strategies to prevent or eliminate infection with tuberculosis and/or to prevent reactivation. It can be used to either replace BCG and/or as a booster to BCG in patients who have already received BCG or another subunit TB immune-stimulant. The vaccine can be used in either prophylactic or therapeutic strategies.

Suitable stimuli for inducing a specific metabolic state include but are not limited to varying oxygen concentrations, carbon monoxide, nutrient availability, presence of nitric oxide, presence of antibiotics, availability of iron, pH changes, Toll-Like Receptor agonists, population density, and/or physical stimulation such as shaking.

Suitable stimuli may be provided in-vitro or in-vivo to the bacilli prior to irradiation.

In some embodiments, 100% of the Mycobacterium spp. cells are inactivated. When the subject is a human, 100% of the Mycobacterium spp. cells are preferably inactivated.

In some embodiments, the Mycobacterium spp. is inactivated with irradiation. Preferably irradiation is with gamma irradiation but other types of radiation may be used including xray and microwaves.

In some embodiments, the Mycobacterium spp. is inactivated with osmotic pressure via salts or a drying process.

The pharmaceutical composition may optionally include an adjuvant to enhance an immune response in the host.

The pharmaceutical composition may optionally include a pharmaceutically acceptable carrier, or be provided lyophilized.

In some embodiments, the pharmaceutical composition is formulated for intranasal delivery to the host.

In addition, the pharmaceutical composition is provided as an aerosol or spray package.

In one embodiment, the invention provides a pharmaceutical composition that includes a gamma-irradiated Mycobacterium spp. that is formulated for intranasal or intrapulmonary delivery to a mammalian host and which confers an immunologically protective dose when delivered to the host, e.g., a human.

In another aspect, the invention provides a method of vaccinating a mammal against TB. The method includes administering to the mammal a composition comprising inactivated Mycobacterium spp. Preferred vaccination is intranasal or intrapulmonary. Preferably, the composition includes an immunologically protective dose when delivered to the host.

In another aspect, the invention provides an immunostimulant that facilitates delivery of another antigen.

In one aspect, the invention provides a pharmaceutical composition comprising an inactivated Mycobacterium spp., wherein the composition is formulated for intranasal, mucosal or intrapulmonary delivery to a mammalian host, and wherein the composition comprises an immunologically protective dose when delivered to the host.

Suitable Mycobacterium spp. for use in the method include, e.g., M. tuberculosis, M. marinum, M. bovis, M. africanum, or M. microtti. In some embodiments, the inactivated Mycobacterium spp. cells are killed cells or cell lysates. When the subject is a human, 100% of the Mycobacterium spp. cells are preferably inactivated.

The pharmaceutical composition for use in the method may optionally include an adjuvant to enhance a protective immune response in the host.

The pharmaceutical composition for use in the method may optionally include a pharmaceutically acceptable carrier, or be provided lyophilized.

In some embodiments, the pharmaceutical composition for use in the method is formulated for intranasal delivery to the host.

In addition, the pharmaceutical composition for use in the method is provided as an aerosol or spray package.

In some embodiments, the pharmaceutical composition is delivered through a device configured for nasal or pulmonary delivery.

In a still further aspect, the invention provides a method for preparing a vaccine for preventing infection with Mycobacterium or treating Mycobacterium infection, comprising formulating an immunologically protective dose of an inactivated Mycobacterium spp. for intranasal or pulmonary delivery to a mammalian host.

In some embodiments, the method includes testing the vaccine in a non-human animal model of tuberculosis. The animal model can be, e.g., a mouse, guinea pig, rabbit, bovine, or non-human primate.

Among the advantages of the invention is that the vaccines disclosed herein emulate the heterogeneous states that are found naturally within the host throughout the bacilli's lifecycle and provide the immune system with multiple states of inactivated bacilli.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

Provided herein are various compositions useful, inter alia, as prophylactic or therapeutic vaccines.

Vaccines Based on Mycobacteria in a Defined State or States

A vaccine according to the invention is prepared using one or more inactivated Mycobacterium spp. in one or more defined metabolic states. The Mycobacterium spp. can then be formulated for delivery to a subject. While not wishing to be bound by theory, Mycobacterium spp. prepared in a defined state is believed to offer advantages to a heterogeneous whole cell preparation. In contrast, a heterogeneous group of inactivated mycobacterium according to the present invention provides a substantial portfolio of mycobacterial antigens to aid the immune system in eliciting a robust memory immune response. The inactivated heterogeneous mycobacterium, when delivered to the lung parenchyma or airway/nasal mucosa of a subject, is postulated to elicit a much stronger immune response than has been observed with previously described TB vaccines.

Defined Mycobacterium States

In general, a specific metabolic state of mycobacterium can be induced by environmental triggers, antibiotics, mycobacterial concentrations, availability of nutrients or presence of oxygen. The species or strain's state may also be induced by gradual changes to nitric oxide, carbon monoxide and pH. A mycobacterium's specific metabolic state may be defined as a sensing and transcription of particular genes as a result of external stimuli. There is ample evidence that gene transcription occurs as a result to different external stimuli. Many researchers have characterized the adaptive mechanisms of M.tb in light of genome data accrued over the last decade. Stationary phase and dormancy gene expression has been characterized as early as 1996 with the characterization of dominant stationary-phase proteins^{[8]}. The gene for the 16-kDa alpha-crystallin-like small heat shock protein was shown to be expressed primarily in the slowly growing M. tb populations. Alternatively, work exploring nutrient starvation of M. tb showed that genes such as Rv2557 and Rv2558 are induced.^{[9]} More recent work also corroborates M.tb's ability to sense carbon monoxide (CO) during macrophage infection and recognizes that the bacterium may be dependent on CO in order to adapt.^{[10]}

M. tb has evolved survival mechanisms to allow for persistent infection and to balance the complex dynamics of the host immune system. M. tb's resilience as a pathogen is largely due to the ability of it to acclimate to environmental factors and sense external stimuli. For example, M.tb can reside in human tissues for years with minimal, if any, replication, and then return to a growth state under appropriate conditions. Research supports the notion that the bacterium's different states are susceptible and triggered by external stimuli, varying environments, chemicals, and antibiotics.^{[11][12][13]}

Various metabolic states of M. tb are thought to be triggered by biosensory mechanisms that alter gene expression to effect intracellular and extracellular status changes. Mycobacterium spp. in specific states are susceptible to inactivation and are postulated to be more effective in eliciting an immune response compared to populations of cells in unknown states or heterogeneous states. The present invention provides Mycobacterium in particular metabolic states.

Successful pathogenesis of M.tb within a host demands an ability to acclimate and adapt to significant challenges. Upon infection into a host, the bacilli are able to survive within macrophages and dendritic cells within the lung and then multiply. The macrophage will phagocytose the bacilli and release lysosomal components (such as inducible nitric oxide synthase and NOX2). Subsequently, the macrophage secretes oxygen and nitrogen intermediates into the phagosome to kill and digest the components.^{[14][15]} M.tb prevents a substantial lowering of pH within the phagosome. Thus, the bacilli avoids a hostile, low pH, environment where it would be incapable of replication.^{[16]} In fact, M.tb within the phagolysosome is thought to help maintain a higher pH of around 6.4 by neutralizing the attempts of the macrophage to lower the pH to less than 5.^{[17]}

Second, in order to control and prevent dissemination, M.tb must be able to persist despite the host's attempts to encapsulate the bacilli within a granuloma. Granulomas are layers of immune cells that surround infected tissues. The center of the granuloma is often composed of dying tissue and macrophages that are fused together. The outside layers of the granuloma include activated macrophages, CD4 and CD8 cells. Bacilli found within the granulomas are often in an environment of low oxygen with high carbon dioxide levels, hydrolytic enzymes, and anti-microbial compounds. Thus, the bacilli's state confers an ability to survive within the granuloma and provides the bacterium an opportunity to reactivate months or even years later.

Third, M.tb can also be found in other organs and tissues of infected individuals where conditions and nutrients may be poor. While the role of this population of bacilli is not thoroughly understood, it is believed that these bacilli might help contribute to persistence and to enable reactivation of the bacilli years later

The sensors and biomechanics of M.tb are now receiving increased attention and more research now highlights the bacilli's sophisticated s mechanisms. In fact, the M. tb's genome encodes about 190 transcriptional regulators of which several have been found to respond to conditions of distress, such as nutritional deprivation, extreme temperatures, iron depletion, and oxidative stress.^{[18]} To survive for a prolonged period in a mammalian host, M. tb adapted to various environmental conditions expression or inhibiting transcription according to its surroundings^{[19]}

In particular, there have been many observations addressing M. tb's ability to withstand anaerobic and aerobic conditions. Tuberculosis bacilli require oxygen for replication but the bacilli can persist for years with extremely low concentrations of oxygen. Rapid changes from aerobic to anaerobic conditions will lead to bacterial death; however, a gradual reduction of O₂ allows for the bacterium to adapt gradually to an environment without O₂.

M. tb can also adapt to in vivo conditions of low iron.^{[12]} An inspection of 22 M. tb genes showed that the whiB3 gene is induced during the early part of M.tb infection. The gene is probably triggered by oxidative and/or reductive stress combined with low bacterial density.^{[20]} Research corroborates that a redox-responsive 4Fe-4S cluster protein specifically reacts with O₂ and NO induces expression of the M. tb WhiB3 gene.^{[21]} The complex responds to physiologically relevant host signals thought to be involved in persistence and latency.^{[21]} In addition, induction of whiB3 is correlated to variation in the intracellular redox environment and is suspected to dependent on aerobic respiration and carbon utilization.

While regulation of whiB3 appears to be complex, the pattern of whiB3 repression in vivo and in vitro is most consistent with regulation by population density.^{[20][21]} Further, expression of whiB3 was found to be inversely correlated with bacterial density in the mouse lung and culture.^{[20]} It is also thought that mycobacterium have an ability to sense mycobacterial population density via the bacteria's cell-cell communication. Ultimately, the ability allows bacteria to adapt as a population or gain population information. Thus, providing either in vivo or in vitro conditions of varying populations may help induce a different state of the mycobacterium.

Another altered state with mycobacterium occurs during oxyben depletion which allows for the bacterium to enter two non-replicating states^{[22]}. The first stage of nonreplicating persistence (NRP1) occurs when the oxygen concentration reaches 1% of normal saturation. This phase, also known as the microaerophilic phase, is characterized by increased optical density within the culture. The cell enlargement is probably a result of a thickening of the cell wall that is usually only observed in hypoxic conditions^{[11]}. This phase is further characterized by RNA synthesis decreases, halting of cell division, and ceasing of DNA synthesis with a host of changes to the enzymatic activities of the mycobacterium. Enzymes include but are not limited to isocitrate lyase, 4 glycine dehydrogenase4 and nitrate reductase.^{[13]} This phase can also be referred to as a stationary phase and refers normally to the cell-density-associated growth arrest in batch cultures caused by oxygen limitation, nutrient limitation, secondary metabolite production and pH changes.^{[23]} It is largely thought that this phase mimics the physiological state exhibited by M.tb during the various stages of persistent infection.

A second, more oxygen-deprived, state, termed non-replicating persistence 2 (NRP2), occurs when the metabolic state of the cell is at a minimum and only necessary functions are active. This second non replicative phase or state occurs when the oxygen level reaches 0.06% of normal saturation (anaerobic). The state is characterized by no further increase in optical density and cessation of cell enlargement. Interestingly, the cells become resistant to antibiotics such as isoniazid and sensitive to antibiotics that treat anaerobes such as metronidazole. Additionally, bacterium in this state can persist for a long period of time. If transferred to appropriate conditions, the bacteria resume growth in a synchronous fashion RNA synthesis begins first, followed by cell division and then finally DNA replication resumes.

One way of inducing NRP2 is to cause a slow depletion of oxygen within a closed system, such as stirred closed culture tube. Initially, cultures are aerobic but as the available oxygen is consumed, the environment shifts into a microaerophilic phase and then into an anaerobic phase. The slow progression allows the bacilli to adapt and survive anaerobic conditions, even though they cannot grow anaerobically.

Rosenkrands et al. indicated than many proteins were such as Rv0569 showed increased levels at 5% oxygen but not at 1% oxygen.^{[24]} The relative abundance of unique proteins investigated using peptide analysis may be much greater than predicted using the NRP model. Moreover, the relative abundance of unique proteins investigated using peptide analysis may be much greater than predicted in the NRP model. Thus, it is clear that finding the appropriate steady states based on protein concentrations assists in providing a suitable mixture of irradiated Mycobacterium spp.

A TB vaccine according to the invention preferably evokes a protective immune response in the mucosal and respiratory mucosal system and preferably directly stimulates the antigen-presenting cells in the respiratory epithelium with various species of mycobacterium in various states.

Inactivation of M. tb

In general, any type of inactivation procedure can be used as long as the treatment leaves the population of bacteria unable to produce a productive infection at the host, while at the same time preserving antigenic structures necessarily for eliciting a productive response to the corresponding disease-causing mycobacterium. The mycobacterium preparation is typically incapacitated. By “incapacitated” in the context of an incapacitated bacterial cell produced according to the invention, is meant that the bacterial cell is in a state of irreversible bacteriostasis. While the bacterium retains its structure—and thus retains, for example, the immunogenicity, antigenicity, and/or receptor-ligand interactions associated with a wild-type bacterium—it is not capable of replicating. In some embodiments, it is incapable of replication due to the presence of an infecting phage with-in the bacterial cell.

A preferred type of inactivation is gamma-irradiation. Other types of inactivation known in the art include, e.g., other types of radiation (including ultra-violet irradiation), formalin treatment, and heat treatment. In the embodiments for human use, 100% of the cells are killed.

While not wishing to be bound by theory, it is postulated that gamma-irradiated Mycobacterium are especially suitable for use in the compositions and methods of the invention. Gamma-irradiated bacteria are commonly used in the laboratory because they are considered safe and do not replicate. In many trials, they have nevertheless been shown to elicit an immunoprotective response, including responses elicited by antigens on the bacilli wall^{[25]}, ^{[26]}, ^{[27]}. In addition, gamma irradiated mycobacterium undergo apoptosis and become engulfed by dendritic cells. Dendritic cells present the mycobacterium antigens to T-cells, which activate CD4 Th1 and CD8 cytotoxic cells. Gamma-irradiated M tb can also induce nitric oxide release^{[25]} and can elicit similar Th2 responses to live M tb^{[26]}. In 1963, Nishihara et al intradermally injected gamma-irradiated M tb into mice and found it was equally as protective as BCG injected intradermally against aerosol challenge with M tb^{[28]}.

The adaptive immune response to live M. tb infection is delayed compared to other infections and this allows the bacilli population in the lungs to markedly increase during the preimmune phase of the infection^{[29]}. By using dead baciili in an aerosolized or mucosal vaccine formulation there is no multiplying mycobacteria and the immune response would have adequate time to respond to the antigens on the cell wall of the bacteria. In addition, over thousands of years through fitness challenges M tb has found many ways to evade the innate immune response during initial antigen presentation^{[30]}, ^{[31]}, ^{[32,33]}. Dead mycobacteria do not have the ability to produce enzymes that evoke ways to evade the human immune system and avoid successful antigen presentation.

Aerosolized or Mucosal Delivery of Vaccine to Pulmonary Tissue

Preferably, Mycobacterium in a desired state are will be used to elicit a localized immune response in the lungs. As the lungs are the initial site of TB infection, it would appear logical to concentrate on a vaccine localized to the pulmonary system. There exists a degree of compartmentalization in the respiratory immune system. Recent evidence suggests that pulmonary lymphocytes remain localized when mounting an initial immune response and only a limited number of B and T-cells migrate systemically^{[34,35]}. The human pulmonary lymph anatomy is unique in that cells entering the thoracic duct from the local pulmonary nodes travel back to the lung in the pulmonary arterial blood before reaching other tissues. Some lymphocytes may pass through to the systemic circulation, but activated T-cells tend to adhere to the vascular endothelium and move back into the lung, thus keeping the T-cells near the foci of infection^{[36]}. In the guinea pig TB model it was observed that pulmonary lymphatics are sites of initial infection in addition to the lungs and regional lymph nodes^{[37][38,39]}. Therefore targeting airway luminal and mucosal immune cells holds important implications for developing effective vaccination strategies. Additionally, airway luminal/mucosally delivered vaccines would have significant advantages such as eliminating the need of needles and enabling rapid vaccination responses in the face of pandemics.

Several studies using aerosol or intra-tracheal delivery of BCG varied in efficacy from superior protection than parenteral inoculation in primates^{[40]}, cattle^{[41]} guinea pigs^{[42]}, and mice^{[43][44,45,46]} to no apparent advantage over the subcutaneous route^{[47]}. Other studies showed immune response was dependent on initial BCG dose^{[48,49]}. Recombinant adenovirus-based vaccines delivered intranasally provided protection against challenge with M. tb^{[39,50,51,52]}. Intranasal immunization of mice with an adenoviral-based vaccine expressing Ag85A^{[53,54]}, a recombinant Streptococcus gordonii expressing Ag85B-ESAT-6^{[55]} or microparticle encapsulated ESAT-6^{[56]} elicited great numbers of antigen-specific CD4+ and CD8+ T cells capable of IFN-γ production. Most recently intranasal delivery of heparin-binding haemagglutinin enhanced protection of BCG-vaccinated newborn mice^{[57]}.

### Compositions

Any Mycobacterium species or strain for which an enhanced immune response is desired can be used in the composition and methods of the invention. Compositions with Mycobacaterium in predefined states can be prepared using procedures described in, e.g., Suitable species include, e.g., Mycobacterium which are members of the M tb complex include, e.g, Mycobacterium bovis, Mycobacterium africanum, Mycobacterium microtti, and Mycobacterium tuberculosis. Mycobacterium that are genetically similar include Mycobacterium canettii and Mycobacterium marinum. The particular species or combination of species is selected for the corresponding host species and type Mycobacterium-associated disease to be treated. Other Mycobacteria that cause disease in humans include, e.g., Mycobacterium avium intracellulare, Mycobacterium leprae, Mycobacterium lepraemurium, Mycobacteria paratuberculosis, Mycobacterium ulcerans, Mycobacterium smegmatis, Mycobacterium xenopi, Mycobacterium chelonei, Mycobacterium fortuitum, Mycobacterium farcinogenes, Mycobacterium flavum, Mycobacterium haemophitum, Mycobacterium kansasii, Mycobacterium phlei, Mycobacterium scrofulaceum, Mycobacterium senegalense, Mycobacterium simiae, Mycobacterium thermoresistible, and Mycobacterium xenopi.

The mycobacterium to be used in the pharmaceutical composition can include whole cells or portions of cells, e.g., cell lysates. For example, suitable components include a gamma irradiated whole cell lysate, gamma irradiated culture filtrate proteins, gamma irradiated cell wall fraction, gamma irradiated cell membrane fraction, gamma irradiated cytosol fraction, gamma irradiated soluble cell wall proteins, and gamma irradiated soluble protein pool.

The mycobacterium to be used in the pharmaceutical composition can include one or more states of mycobacterium whether in whole cells or portions of cells, e.g. cell lysates.

### Preparing Pharmaceutical Compositions

The killed cells are prepared for administration to a host by combining inactivated cells in a desired state or states, or cell lysates with a pharmaceutically acceptable carrier to form a pharmaceutical composition. The carrier can be, e.g., such as physiological saline, mineral oil, vegetable oils, aqueous sodium carboxymethyl cellulose, or aqueous polyvinylpyrrolidone. In some embodiments, the carrier is sufficiently pure to be administered therapeutically to a human subject. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants and/or other additives may be included, as required.

A skilled person in the field familiar with the protocols, formulations, dosages and clinical practice associated with, e.g., the administration of M. bovis BCG can in addition readily adapt these protocols for use with pharmaceutical compositions of the present invention. The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 1000 mg. Suitable regimens for initial administration and booster shots are also variable but are typified by an initial administration followed by subsequent inoculations or other administrations. Thus, the vaccine may be administered in a single dose or in a plurality of doses. In one embodiment, the vaccine may be administered in two doses about 1-12 months apart. The subject may be vaccinated at any time, although it is preferred to administer the vaccine shortly (optimally about 10 days to two weeks) before periods of anticipated stress, such as during shipping or other handling.

A composition may be administered alone or in combination with other treatments or standard BCG vaccine, either simultaneously or sequentially dependent upon the condition to be treated. The composition can be administered after vaccination with BCG and therefore act as a boosting tuberculosis vaccine. Moreover, it may be given after an initial subcutaneous inoculation of the whole killed bacilli followed by an aerosolized, intranasal or mucosal boost.

The killed cells may be incorporated into microparticles or microcapsules to prolong the exposure of the antigenic material to the subject animal and hence protect the animal against infection for long periods of time. The microparticles and capsules may be formed from a variety of well-known inert, biocompatible matrix materials using techniques conventional in the art. Suitable matrix materials include, e.g., natural or synthetic polymers such as alginates, poly(lactic acid), poly(lactic/glycolic acid), poly(caprolactone), polycarbonates, polyamides, polyanhydrides, polyortho esters, polyacetals, polycyanoacrylates, polyurethanes, ethyllene-vinyl acetate copolymers, polystyrenes, polyvinyl chloride, polyvinyl fluoride, poly(vinyl imidazole), chlorosulphonated polyolefins, polyethylene oxide, and particularly agar and polyacrylates. Examples of techniques for incorporation of materials into microparticles or encapsulation which may be used herein are described by Sparks^{[58]}, Kydonius^{[59]}, and El-Nokaly^{[60]} the contents of each of which are incorporated by reference herein.

The inactivated mycobacterium may be contained in small particles suspended in water or saline. The vaccine formulations may also contain optional adjuvants, antibacterial agents or other pharmaceutically active agents as are conventional in the art. Adjuvants may include but are not limited to salts, emulsions (including oil/water compositions), saponins, liposomal formulations, virus particles, polypeptides, pathogen-associated molecular patterns (PAMPS), nucleic acid-based compounds or other formulations utilizing certain antigens. Suitable adjuvants include, e.g., vegetable oils, alum, Freund's incomplete adjuvant, or Freund's incomplete adjuvant, with oils and Freund's incomplete adjuvant being particularly preferred. Other adjuvants include agents such as aluminum hydroxide or phosphate (alum), immune-stimulating complexes (ISCOMs), synthetic polymers of sugars (CARBOPOL®), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

The inactivated mycobacterium may be contained in a mucosal bacterial toxin adjuvant such as the Escherichia coli labile toxi (LT) and cholera toxin (CT) or in CpG oligodeoxynucleotide (CpG ODN)^{[61]}. Another possible mucosal adjuvant Monophosphoryl lipid A (MPL), a derivative and less toxic form of LPS, when combined with liposomes was found to induce mucosal immunoprotective responses^{[62]}. One new adjuvant designed for nasal vaccination, Eurocine L3™, has been shown to induce long-lasting immunity against TB in experimental animal models after intranasal administration^{[63,64,65]}. The adjuvant technology consists of a non-toxic pharmaceutical formulation based on a combination of endogenous and pharmaceutically accepted lipids. The vaccine may optionally include additional immune modulating substances such as cytokines or synthetic IFN-γ inducers such as poly I:C alone or in combination with the above-mentioned adjuvants.

Still other adjuvants include microparticles or beads of biocompatible matrix materials. The microparticles may be composed of any biocompatible matrix materials as are conventional in the art, including but not limited to, agar and polyacrylates. The practitioner skilled in the art will recognize that other carriers or adjuvants may be used as well. For example, Chitosan or any bioadhesive delivery system which may be used are described by Webb and Winkelstein^{[66]} the contents of which are incorporated by reference herein.

The composition optionally includes Vitamin D, and/or its metabolites, analogues or its derivatives as part of the aerosolized dose. A person skilled in the art will recognize that Vitamin D may assist in triggering of toll like receptors.

The pharmaceutical composition containing the inactivated mycobacterium is preferably formulated for intranasal or intrapulmonary delivery using methods known in the art. The formulation of the irradiated mycobacterium combined with the adjuvant is preferably selected to minimize side effects, such as inflammation, associated with vaccination or may improve the formulation's stability. The adjuvant may also have a role as an immunostimulant or as a depot. In order for deep lung penetration, particle size is preferably between 1-4 microns.

In some embodiments, the inactivated mycobacterium are delivered by the refinement of a nebulizer or via three types of compact portable devices, the metered-dose inhaler (MDI) and the dry powder inhaler (DPI). Intransal delivery can occur via the nasal spray, dropper or nasal metered drug delivery device. The inactive mycobacterium may be delivered via a metered dose inhaler. Typically, only 10-20% of the emitted dose is deposited in the lung. The high velocity and large particle size of the spray causes approximately 50-80% of the drug aerosol to impact in the oropharyngeal region.

The mycobacterium may be contained in a dry powder formulation such as but not limited to a sugar carrier system. The Sugar Carrier System could include lactose, mannitol, and/or glucose. Lactose, mannitol, and glucose are all approved by the FDA as carriers. There are also larger sugar particles such as lactose monohydrate-typically 50-100 micrometers in diameter, which remain in the naso-oropharynx but allows the inactivated bacilli to travel through the respiratory tree into the alveoli.^{[67]}

If desired, the mycobacterium may be contained in a liposomal formulation. Liposomes, like other inhaled particles reaching the alveoli, are cleared by macrophages. The processing, uptake and recycling of liposomal phospholipids occurs through the same mechanism as endogenous surfactant via the alveolar type II cells.

### Terminology

A pharmaceutical composition containing the irradiated mycobacterium described above is administered to a suitable individual for preventing or treating tuberculosis. Reference herein to “tuberculosis” includes reference to pulmonary and extra-pulmonary bacilli. The terms “individual,” “subject,” “host,” and “patient,” are used interchangeably herein and refer to any subject having a bacterial infection amenable to treatment using the therapeutic vaccine of the invention, and for whom treatment or therapy is desired. The pharmaceutical composition can be prepared for any mammalian host that is susceptible to infection by mycobacterium. Suitable mammalian hosts include, e.g., farm animals such as swine and bovine

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom (c) preventing reactivation of the disease in latent TB, i.e. preventing the bacilli from transitioning from a dormant to growth phase. Thus, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical or veterinarian.

The subject treated with the vaccine typically will have or will develop protective immunity to an infecting bacterium. The term “protective immunity” means that a vaccine, immunogenic composition or immunization schedule that is administered to a mammal induces an immune response that prevents, retards the development of, or reduces the severity of a disease that is caused by a pathogenic bacterium or diminishes or altogether eliminates the symptoms of the disease. By “infecting bacterium” is meant a bacterium that has established infection in the host, and which may be associated with a disease or undesirable symptom as a result. Generally, infecting bacteria are pathogenic bacteria.

The phrase “in a sufficient amount to elicit an immune response” means that there is a detectable difference between an immune response indicator measured before and after administration of a particular vaccine preparation or immunogenic composition. Animals given the vaccine trial will be tested against animals given intradermal BCG. Several weeks after the last vaccination, animals will be challenged with aerosol virulent M tb. The clinical and molecular immune response is evaluated several weeks after challenge with virulent M. tb.

The term “state”, “metabolic state”, “altered state”, or “different state” might be used interchangeably and refer to different metabolic states of the mycobacterium in which there is a detectable difference in protein composition or gene expression. A state is defined whereby a bacterium has responded to external stimuli and as a result the bacterium presents different antigens or may express and begin transcription of particular genes.

### Screening and Developing Tuberculosis Vaccines

A test vaccine can be screened or optimized by subjecting a population of mycobacterium cells, or fractions thereof (as described above) to various inactivation regimens, preparing a candidate pharmaceutical composition containing the treated cells or cell fractions and testing the ability of the treated composition using the methods described above to elicit an immune response and/or mount an effective challenge to mycobacterium infection in a host.

The terms “immunogenic bacterial composition”, “immunogenic composition”, and “vaccine” are used interchangeably herein to mean a preparation capable of eliciting a cellular and/or humoral immune response in a subject when administered in a sufficient amount to elicit an immune response to epitopes present in said preparation.

The terms “state” or “phase” are used interchangeably herein and refer to the mycobacterium's metabolic state in response to external stimuli or environment.

Immunopotency of the antigenic molecule expressed by the mycobacterium cell or extract preparation, can be determined by monitoring the immune response of test animals following immunization with the bacteria expressing the recombinant antigen. Test animals may include mice, guinea pigs, rabbits, bovine, non-human primates, and eventually human subjects.

The immune response of the test subject can additionally be analyzed by various approaches such as: (a) T-cell associate cytokine production (b) plasma cytokine production (c) T cell proliferation, cytoxicity, cytokine profiles (d) T cell antigen repertoire (e) T cell regulatory profiles (f) mRNA profiles (g) innate immunity profiles (h) antibody profiles (i) genetics and (j) protection from disease and/or mitigation of infectious symptoms in immunized animals.

### Bacterium Composition and Method of Using Same

In another aspect the invention relates to a vaccine against tuberculosis and more particularly to a vaccine using inactivated Mycobacterium spp. formulated for oral, rectal, or vaginal delivery to subject.

Mucosal vaccination and therapy represents the next frontier in immunology. The gut associated lymphoid tissue and its immune system represent a significant challenge for treating or vaccinating against Mycobacterium species and or other bacteria such as Brucella species. Mycobacterium species and Brucella species are resilient intracellular bacterium that have co-evolved with humans and continue to impact humans and livestock. Likewise in cattle, Brucellosis and Johne's disease represent significant problems to livestock health and represent significant financial burdens to agriculture. Current vaccines for these two diseases offer questionable efficacy and raise safety concerns. Additionally, Crohn's disease remains without a vaccine alternative. A new approach is needed for inducing an immune response within the gut associated lymphoid tissue and reducing the disease prevalence caused by these intracellular pathogens.

The invention provides a composition for a vaccine for preventing and/or treating bacterial borne disease, e.g., disease caused by inactivated mycobacterium species and inactivated bacteria.

The composition can be used with a number of vaccination strategies: prophylactically, given prior to infection to prevent infection with bacteria and therapeutically, when it is administered post-exposure to eliminate or contain latent and prevent reactivation. The composition can also be used as a treatment for a bacterial, viral or fungal infection or an autoimmune disease. Vaccines using a composition of the invention can be used to replace a current vaccine and/or as a booster to other vaccines in a subject.

The composition can be formulated for oral, rectal or vaginal delivery. Also within the invention is an osmotic delivery system and other formulations delivery systems for controlling the rate of delivery of the antigenic material in order to maximize exposure to mucosal tissue such as the gut mucosal lymphoid tissue.

The formulation may be used as part of a composition containing bacteria or viral components, either as whole entities or as partial components. The localized delivery of irradiated mycobacterium to mucosal surfaces of the intestines or reproductive system can act as an adjuvant and/or or therapeutic agent.

In one aspect, the invention provides a pharmaceutical composition comprising one or more mycobacterium species, wherein the composition is formulated for vaginal, rectal, or oral delivery to a mammalian host, and wherein the composition comprises an immunologically protective dose when delivered to the host.

In one aspect, the invention provides a vaccination or therapeutic for autoimmune disease in cattle such as Johne's disease, inflammatory bowel disease or Crohns disease to a mammal.

The composition might include irradiated and/or inactivated mycobacterial species (spp). Suitable Mycobacterium spp. include, e.g., Mycobacterium avium subspecies paratuberculosis, M. tuberculosis, M. marinum, M. bovis, M. africanum, or M. microtti. In some embodiments, the inactivated Mycobacterium spp. cells are killed cells or cell lysates.

In general, any Mycobacterium species or strain that is a member of the M. tuberculosis complex can be used in the composition and methods of the invention. The particular species or combination of species is selected for the corresponding host species and type Mycobacterium-associated disease to be treated. Thus, Mycobacteria that cause disease in humans include, e.g., Mycobacterium avium intracellulare, Mycobacterium leprae, Mycobacterium lepraemurium, Mycobacteria paratuberculosis, Mycobacterium ulcerans, Mycobacterium smegmatis, Mycobacterium xenopi, Mycobacterium chelonei, Mycobacterium fortuitum, Mycobacterium farcinogenes, Mycobacterium flavum, Mycobacterium haemophitum, Mycobacterium kansasii, Mycobacterium phlei, Mycobacterium scrofulaceum, Mycobacterium senegalense, Mycobacterium simiae, Mycobacterium thermoresistible, and Mycobacterium xenopi. Mycobacterium, Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium tuberculosis, Suitable Mycobacterium species, which are members of the M tb. complex include: Mycobacterium bovis, Mycobacterium africanum, Mycobacterium microtti, and Mycobacterium tuberculosis. Genetically-similar mycobacterium include Mycobacterium canettii and Mycobacterium marinum.

Additional suitable bacteria include, e.g., Acetobacter aurantius, Acinetobacter baumannii, Actinomyces israelii, Agrobacterium radiobacter, Agrobacterium tumefaciens, Azorhizobium caulinodans, Azotobacter vinelandii, Anaplasma, Anaplasma phagocytophilum, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaminogenicus, Bartonella, Bartonella henselae, Bartonella quintana, Bordetella, Bordetella bronchiseptica, Bordetella pertussis, Borrelia burgdorferi, Brucella, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila, Chlamydophila pneumoniae, Chlamydophila psittaci, Clostridium, Clostridium botulinum, Clostridium difficile, Clostridium perfringens, Clostridium tetani, Corynebacterium, Corynebacterium diphtheria, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenza, Haemophilus parainfluenzae, Haemophilus pertussis, Haemophilus vaginalis, Helicobacter pylori, Klebsiella pneumonia, Lactobacillus, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycoplasma fermentans, Mycoplasma genitalium, Mycoplasma hominis, Mycoplasma penetrans, Mycoplasma pneumonia, Lactobacillus Bulgaricus, Neisseria, Neisseria gonorrhoeae, Neisseria meningitides, Pasteurella, Pasteurella multocida, Pasteurella tularensis, Peptostreptococcus, Porphyromonas gingivalis, Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumonia, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholera, Vibrio comma, Vibrio parahaemolyticus, Vibrio vulnificus, Wolbachia, Yersinia, Yersinia enterocolitica, Yersinia pestis, Yersinia pseudotuberculosis.

In some embodiments, the bacteria are killed cells or cell lysates.

In some embodiments, some of the bacteria are inactivated or attenuated.

In some embodiments, the bacteria are inactivated with irradiation. Preferably irradiation is with gamma irradiation but other types of radiation may be used, including xray and microwaves.

In some embodiments, the bacteria are inactivated with osmotic pressure via salts or drying process.

The pharmaceutical composition may optionally include an adjuvant to enhance an immune response in the host.

The pharmaceutical composition may optionally include a pharmaceutically acceptable carrier, or be provided lyophilized.

The pharmaceutical composition may optionally include a pharmaceutically acceptable carrier such as glucose, lactose or sorbitol.

In some embodiments, the pharmaceutical composition is formulated for oral, rectal or vaginal delivery to the host.

In addition, the pharmaceutical composition may be provided as a suppository package or in an oral formulation.

In one embodiment, the invention provides a pharmaceutical composition that includes a gamma-irradiated Mycobacterium spp. that is formulated for oral, rectal or vaginal or intrapulmonary delivery to a mammalian host and which confers an immunologically protective dose when delivered to the host, e.g., a human.

In another aspect, the invention provides a method of vaccinating a mammal against TB. The method includes administering to the mammal a composition comprising inactivated Mycobacterium spp., wherein the vaccination of the mammal is oral, rectal or vaginal, and wherein the composition comprises an immunologically protective dose when delivered to the host.

In another aspect, the invention provides an immunostimulant that facilitates delivery of another antigen.

In one aspect, the invention provides a pharmaceutical composition comprising a carrier such as a osmotic delivery device or matrix composition and gamma irradiated Mycobacterium spp., wherein the composition is formulated for gastrointestinal delivery to a mammalian host, and wherein the composition comprises an immunologically protective dose when delivered to the host.

In some embodiments, the inactivated Mycobacterium spp. cells are killed cells or cell lysates. When the subject is a human, 100% of the Mycobacterium spp. cells are preferably inactivated. In some embodiments, the Mycobacterium spp. for use in the method is inactivated with irradiation. Preferably irradiation is with gamma irradiation. In other embodiments, the Mycobacterium spp. is inactivated with formalin or heat.

In one aspect, the invention provides a pharmaceutical composition comprising a carrier such as a osmotic delivery device or matrix composition and inactivated Brucella abortus, wherein the composition is formulated for gastrointestinal delivery to a mammalian host, and wherein the composition comprises an immunologically protective dose when delivered to the host.

In some embodiments, the Brucella abortus cells are killed cells or cell lysates. When the subject is a human, 100% of Brucella spp. cells are preferably inactivated. In some embodiments, the Brucella spp. for use in the method is inactivated with irradiation. Preferably irradiation is with gamma irradiation. In other embodiments, the Brucella spp. is inactivated with formalin or heat.

The pharmaceutical composition for use in the method may optionally include an adjuvant to enhance a protective immune response in the host.

The pharmaceutical composition for use in the method may optionally include a pharmaceutically acceptable carrier, or be provided lyophilized.

In some embodiments, the pharmaceutical composition for use in the method is formulated for delivery to the gut associated lymphoid tissue.

In some embodiments, the pharmaceutical composition is delivered through a device designed for delivery into the gastrointestinal system.

In a still further aspect, the invention provides a method for preparing a vaccine for treating Mycobacterium infection, comprising formulating an immunologically protective dose of an inactivated Mycobacterium spp. for gastrointestinal delivery to a mammalian host.

In a still further aspect, the invention provides a method for preparing a vaccine for treating Brucella abortus infection, comprising formulating an immunologically protective dose of an inactivated Brucella abortus spp. for gastrointestinal delivery to a mammalian host.

In some embodiments, the method includes testing the vaccine in a bovine animal model of Johne's disease, Crohn's Disease or Brucellosis. The animal model can also be other mammals such as be, e.g., a mouse, guinea pig, rabbit, swine, goat, or deer.

The immune system of a mammal's gastrointestinal system is often referred to as gut-associated lymphoid tissue (GALT). This important system includes the intestine, which contains the largest mass of lymphoid tissue in the human body. GALT helps protect a mammal from foreign matter such as bacteria and viruses via many types of lymphoid tissue that store immune cells. Immune cells, such as T and B lymphocytes, are responsible for defending the body from foreign invaders. The network of tissues throughout the gastrointestinal system include: tonsils (Waldeyer's ring), adenoids (Pharyngeal tonsils), Peyer's patches, lymphoid aggregates in the appendix and large intestine and stomach, small lymphoid aggregates in the esophagus, and lymphoid cells and plasma cells in the lamina propria of the gut.

The lumen of the gastrointestinal tract represents the external world to the body. The immune system is able to isolate foreign matter via the mucosal lining and with a host of cells such as lymphocytes, macrophages and other cells. The population of lymphocytes within the GALT is similar to that of the spleen and are largely described as lamina propria lymphocytes, itraepithelial lymphocytes, and Peyer's Patches. Located in the mucosa and submucosa of the small intestine, Peyer's Patches are similar to lymph nodes that can be found throughout the intestinal tract. Lastly, M or microfold cells are located in the intestinal epithelium over lymphoid follicles and endocytose protein and peptide antigens. M cells transport foreign matter into the underlying tissue, to be passed to local dendritic cells and macrophages. Dendritic cells and macrophages then present T cells in the GALT. Moreover, dendritic cells below the epithelium can also detect antigens in the lumen with by with pseudopods located between epithelial cells. Following exposure to antigens in Peyer's patches, T cells migrate into the lamina propria and the epithelium, where they undergo maturation.

A new generation of oral vaccines with delivery systems that target antigens to gut-associated lymphoid tissue would revolutionize treatment of gastrointestinal diseases, immunotherapy, and therapeutics. The present compositions and methods use the potential of microfold (M) cells by emulating the entry of pathogens into these cells. The stimulation of M cells by the proposed composition might enhance the entry of antigens, initiating the immune response and consequently leading to protection against mucosal pathogens.

The present invention delivers inactivated mycobacterium or another bacteria to the gut associated lymphoid tissue in order to elicit an immune response. The compositions and methods account for the environment, immunological target and transition times within the stomach (<3 hr), small intestine (3-5 hr), and large intestine (20 hr).

In order to provide for ideal delivery, environmental factors such as pH, osmolarity, and biochemical milieu are considered. For example, within pH occurs within a dynamic range without the gastrointestinal tract: Stomach (before meal) 1-2, Stomach (during digestion) 4, Small intestine 6-7, Duodenum 6.6, Ileum 7.5 and Cecum 6.4. Such changes in pH could constitute a substantial degradation to any composition including inactivated mycobacterium. The present invention uses osmotic delivery vehicles or matrix compositions to deliver the mycobacterium to sites beyond the stomach, where the pH is more amenable to the mycobacterial antigen stability and presentation. Moreover, the delivery of drugs to the lower gastrointestinal tract is advantageous for localized therapy of colonic diseases such as inflammatory bowel disease (Crohn's disease and ulcerative colitis), irritable bowel syndrome, and colon cancer.

While not wishing to be bound by theory, it is believed that localized delivery of antigen enhances the efficacy of a vaccination or immunotherapy. Analogous insights in other organs such as the lung demonstrate the importance of localized delivery and the induction of important homing mechanisms. For example, it has been found in the murine TB model that antigen specific memory T-cells in the lungs preferentially home back to the site of vaccination and that the location of T cells in the airway at the time of infection is of importance. Moreover, research in an influenza murine model suggests that pulmonary immune cells remain localized and only a few B cells and T cells migrate systemically. Based on proprietary data together with these aforementioned insights, the inventors hypothesize a benefit derived from the homing mechanism of the immune system. Applying these findings to the instant invention, then, for a mycobacterial vaccine to be successful in evoking a protective immune response in the gut associated lymphoid tissue, it preferably directly stimulates the antigen-presenting cells within the GALT. The invention accomplishes this by delivering irradiated mycobacterium or other bacteria directly to the large and small intestines with the use of the applications and delivery systems set forth.

The compositions and methods of the invention are useful for diseases and conditions including, e.g., Crohn's disease and livestock diseases such as Johne's disease and Brucella abortus.

Human Crohn's Disease

MAP has also been linked to Crohn's disease, an inflammatory bowel disease in humans as it causes a very similar disease, Johne's disease, in cattle. While many pathogenic bacteria have been suspected of being causative agents of Crohn's disease, most research supports that a weakened mucosal layer and inability to clear bacteria from the intestinal walls are allowing safer harbor for bacteria. Moreover, there is evidence that Crohn's disease, ulcerative colitis and irritable bowel syndrome may have the same underlying cause. In fact, the concurrence of Crohn's disease, Mycobacterium, other bacteria, and genetic markers has recently observed and it is now known that many individuals may have genetic factors that predispose them to Mycobacterium avium subsp. paratuberculosis infection. Upon infection in humans, the bacterium produces mannins, which protect both itself and various bacteria from phagocytosis, which causes a variety of secondary infections. Approximately, 1.4 million Americans suffer from inflammatory bowel diseases, or IBD including Crohn's disease and ulcerative colitis.

Use in Johne's Disease

Johne's disease, an infectious bacterial disease most commonly affects livestock such as cattle, sheep, and goats and has been reported in other species of captive and wild species of ruminants. Caused by the bacterium Mycobacterium avium subspecies paratuberculosis, Johne's disease is often referred to by the acronym MAP. Mycobacterium, including MAP, is an intracellular bacterium capable of growing and replicate inside cells of an animal's immune system. MAP contaminates water, soil and plants when it is excreted by infected animals in feces, milk and saliva. While the bacterium is not as robust in an external environment, MAP is capable of surviving as long as a year due to its ability to acclimate to a wide range of temperature, as well as changes in pH and water availability. Uninfected animals that are exposed to feces, saliva or other contaminated sources are at high risk for the disease.

Infected animals may be asymptomatic for years after the initial infection. However, symptomatic animals will experience prolonged diarrhea and weight-loss. The disease manifests itself in four stages: Stage 1 is described as asymptomatic with shedding of MAP via excretions. Stage II is described by an asymptomatic animal with more significant shedding that presents a substantial increased risk to other surrounding animals. Diagnosis is typically only detectable in feces. Stage III is the beginning of noticeable symptoms and most diagnostic assay can detect the mycobacterium's presence. Stage IV is clinically obvious where the animal may shed billions of bacterium per day, and may not occur for as much as two years or longer. Given its contagious nature, Johne's disease may only present in a few animals at first and it is thought that as many as 5-15 times the number of animals may be infected by first diagnosis.

Another means of transmission is from milk from infected females. Research indicates that 36% of cows with Johne's in Stage III and IV have bacterium in the colostrum. Nursing calves have a high degree of likelihood to pass infection via the colostrum, milk or exposure to contaminated areas outside of teats. Risk of prenatal infection is from 8 to 40% if the mother is in stage III or IV but the risk is much lower in mothers with Stages I and II infection.

According to a dairy study by the National Animal Health Monitoring System (NAHMS), Johne's disease is percent in 22% of U.S dairies whereby a positive finding required greater than 10% of cows test positive for MAP. It is estimated that the cost to U.S dairy industry is greater than $200 million annually via reduced milk production, reduction of slaughter value and premature culling. Even though the vaccination against MAP does not protect against infection and did not prevent losses of milk production, it is considered marginally effective to reduce shedding and clinical symptoms. Vaccination against paratuberculosis has been reported to be was highly profitable with an economic profit of $142 per cow post vaccination. Due to lack of efficacy, vaccination is the least commonly utilized strategy for reducing MAP infection. Two other strategies employed beyond vaccination are test and cull as well as quarantine to reduce susceptible calves or livestock.

Use in Bovine Brucella abortus

Brucellosis is caused by Brucella abortus and can lead to severe illness and death in livestock and humans. Brucellosis in livestock can result in abortions of the infected livestock. Since positive diagnosis can result in slaughter of reactive animals, the testing and slaughter pose an economic threat for the U.S. cattle industry. While the United States and Western Europe bear an economic burden from the disease, Brucellosis remains a significant health threat in Africa, the Middle East, South America, and other developing areas of the world. While the disease is chronic and asymptomatic, pregnant heffers may suffer from a placental infection that can lead to abortion and reduced fertility. Brucella abortus in wildlife is a continuous threat to livestock and states such as Montana, Idaho and Wyoming have all reported recent exposures.

Brucella abortus have evolved mechanisms that resist killing by neutrophils following phagocytosis, replicate inside macrophages and provide escape mechanisms from the macrophage. Thus development of a vaccine technology has been difficult to thwart the worthy adversary. B. abortus RB51 and B. melitensis REV.1 are used to immunize livestock in many countries; however, the strains still induce abortion and persistent infection. Moreover, the REV.1 vaccine is virulent and unstable, creating the need for improved vaccines for B. melitensis. At best, the current vaccines have less than 60% efficacy even after revaccination and the efficacy in wildlife is less. Thus, with questions of efficacy and safety, research beyond the current vaccines is needed.

Preparing Pharmaceutical Compositions

A vaccine according to the invention is prepared using one or more inactivated Mycobacterium spp. or other bacteria that is then formulated for rectal, oral or vaginal delivery to a subject. The inactivated mycobacterium or bacteria, when delivered to the small intestines mucosal and large intestines mucosa of a subject is postulated to elicit a strong immune response.

The composition may be delivered as part of a feed regimen or delivered in conjunction with specialized plant based vaccines or seed crops such as rice, maize, or soybeans.

In another embodiment, the composition is prepared for administration to a host by combining with a pharmaceutically acceptable carrier to form a pharmaceutical composition.

Alternatively, the composition is prepared for gastric delivery with the use of pH-sensitive polymers that enhance gastric release, mucoadhesive polymers for gastric retention and release, or gastric retention systems.

The composition in one embodiment is prepared for enteric delivery with pH-sensitive polymers that resist gastric dissolution, swelling/gelling HG for controlled release, or osmotic pressure-driven tablet or device for controlled.

In some embodiments, the composition is prepared for colonic delivery with the use of compositions that may be degradable by colonic bacteria such as azoreductases, esterases, amidases, glucosidase, glucuronidase.

If desired, the composition is prepared for colonic delivery with the use of compositions that utilize osmotic or swelling systems that release at times well beyond gastric and/or enteric transit times.

The composition can be coated if desired with suitable polymers which degrade only in the colon or intestines.

The inventors hypothesize that the use of calcitriol delivered concurrently with inactivated mycobacterium or Brucella abortus has beneficial properties. It is envisioned that oral, rectal or vaginal delivery of inactivated mycobacterium in conjunction with a form of Vitamin D (calcitriol) would aid in the engulfment and processing of the bacterium to allow for macrophage antigen presentation and imbue an enhanced immune response. The calcitriol stimulates catheliciden within the macrophage vacuoles to kill and disassemble the bacteria's antigenic components. Vitamin D has been linked with Toll-Like Receptor signaling and presentation of macrophages with vitamin D-1-hydroxylase may induce expression of an anti-microbial peptide cathelicidin to promote sufficient killing of mycobacteria

A further enhancement is to use different metabolic states of mycobacterium added to a Vitamin D composition. This has the potential of improving mycobacteriumantigen presentation to the cellular immune response

The composition is optionally coated with pH-sensitive polymers which take advantage of the progressive increase in the pH from stomach to distal ileum. The coating of pH sensitive polymers to the tablets, capsules or pellets provides protection from the acidic gastric fluid and may include but is not limited to Eudragit L 100, Eudragit S 100, Eudragit L 30 D, Eudragit FS 30 D, Eudragit L 100-55, Polyvinyl acetate phthalate, Hydroxypropyl ethylcellulose phthalate, Hydroxypropyl ethylcellulose phthalate 50, Hydroxypropyl ethylcellulose phthalate 55, Cellulose acetate trimelliate, and Cellulose acetate phthalate.

Suitable osmotic delivery devices include, e.g., a Rose Nelson pump, a Higuchi Leeper pump, a Higuchi Theeuwes pump, an elementary osmotic pump, a multichamber osmotic pump, an OROS-CT. Other devices include, e.g., multi particulate delayed release systems, Liquid Oral Osmotic System, Sandwiched osmotic tablet, a Monolithic osmotic system, an osmotic bursting osmotic pump, or a Telescopic capsule for delayed release. Other systems include, e.g., pulsatile delivery by a series of stops, pulsatile delivery based on expandable orifice, liquid osmotic pump or push pull pump.

The bacterium to be used in the pharmaceutical composition can include whole cells or portions of cells, e.g., cell lysates. For example, suitable components include a gamma irradiated whole cell lysate, gamma irradiated culture filtrate proteins, gamma irradiated cell wall fraction, gamma irradiated cell membrane fraction, gamma irradiated cytosol fraction, gamma irradiated soluble cell wall proteins, and gamma irradiated soluble protein pool. Preservatives, stabilizers, buffers, antioxidants and/or other additives may be included, as required.

A skilled person in the field familiar with the protocols, formulations, dosages and clinical practice associated with, e.g., the administration of inactivated mycobacterium or bacteria can in addition readily adapt these protocols for use with pharmaceutical compositions of the present invention. The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 1000 mg. Suitable regimens for initial administration and booster shots are also variable but are typified by an initial administration followed by subsequent inoculations or other administrations. Thus, the vaccine may be administered in a single dose or in a plurality of doses. In one embodiment, the vaccine may be administered in two doses about 1-12 months apart. The subject may be vaccinated at any time, although it is preferred to administer the vaccine shortly (optimally about 10 days to two weeks) before periods of anticipated stress, such as during shipping or other handling.

A composition may be administered alone or in combination with other treatments or standard vaccine, either simultaneously or sequentially dependent upon the condition to be treated. The composition can be administered after vaccination with and therefore act as an adjuvant for a vaccine.

In another embodiment, the composition of the invention will compensate for differential pH or absorption in the gastrointestinal tract. For example, the composition may have specific concentrations of the release modifier or the active ingredient in different zones of a matrix in order to provide different rates of release of the active substance in the small intestine and in the large intestine. A person skilled in the art will be able to determine the appropriate composition and may employ routine testing to determine the appropriate testing.

The composition may utilize a dual staged release targeting the small and large intestine. In this embodiment, the composition would contain a release modifier that is soluble with the pH of the small and large intestines respectively. Since the small intestine and large intestine typically having a pH of 7.1-7.2 and 6.9 respectively, a release modifier which is soluble at a pH above 7.0 or 7.1, and insoluble below 7.0, may be chosen.

The composition may be contained in small particles suspended in the water or saline. The composition may also contain additional adjuvants, antibacterial agents or other pharmaceutically active agents as are conventional in the art. Adjuvants may include but are not limited to salts, emulsions (including oil/water compositions), saponins, liposomal formulations, virus particles, polypeptides, pathogen-associated molecular patterns (PAMPS), nucleic acid-based compounds or other formulations utilizing certain antigens. Suitable adjuvants include, e.g., vegetable oils, alum, Freund's incomplete adjuvant, or Freund's incomplete adjuvant, with oils and Freund's incomplete adjuvant being particularly preferred. Other adjuvants include agents such as aluminum hydroxide or phosphate (alum), immune-stimulating complexes (ISCOMs), synthetic polymers of sugars (CARBOPOL®), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be used.

The composition may optionally be contained in a mucosal bacterial toxin adjuvant such as the Escherichia coli labile toxi (LT) and cholera toxin (CT) or in CpG oligodeoxynucleotide (CpG ODN. Another possible mucosal adjuvant Monophosphoryl lipid A (MPL), a derivative and less toxic form of LPS, when combined with liposomes was found to induce mucosal immunoprotective responses. The adjuvant technology consists of a non-toxic pharmaceutical formulation based on a combination of endogenous and pharmaceutically accepted lipids. The vaccine may optionally include additional immune modulating substances such as cytokines or synthetic IFN-γ inducers such as poly I:C alone or in combination with the above-mentioned adjuvants.

Still other adjuvants include microparticles or beads of biocompatible matrix materials. The microparticles may be composed of any biocompatible matrix materials as are conventional in the art, including but not limited to, agar and polyacrylates. The practitioner skilled in the art will recognize that other carriers or adjuvants may be used as well. For example, chitosan or any bioadhesive delivery-system that can be used are described by Webb and Winkelstein the contents of which are incorporated by reference herein.

The pharmaceutical composition is preferably formulated for vaginal, rectal or oral delivery using methods known in the art. The formulation of the irradiated composition combined with the adjuvant is preferably selected to minimize side effects, such as inflammation, associated with vaccination or may improve the formulation's stability. The adjuvant may also have a role as an immunostimulant or as a depot.

If desired, the composition may be contained in a liposomal formulation. Liposomes, like other particles reaching the alveoli, are cleared by macrophages. The processing, uptake and recycling of liposomal phospholipids occurs through the same mechanism as endogenous surfactant via the alveolar type II cells.

A pharmaceutical composition containing the irradiated mycobacterium described above is administered to a suitable individual for preventing or treating tuberculosis. Compositions can be made using methods disclosed in Lighter et al., US20100112007. Reference herein to “tuberculosis” includes reference to pulmonary and extra-pulmonary tuberculi. The terms “individual,” “subject,” “host,” and “patient,” are used interchangeably herein and refer to any subject having a bacterial infection amenable to treatment using the therapeutic vaccine of the invention, and for whom treatment or therapy is desired. The pharmaceutical composition can be prepared for any mammalian host that is susceptible to infection by mycobacterium. Suitable mammalian hosts include, e.g., farm animals such as swine and bovine.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom (c) preventing reactivation of the disease, i.e. preventing the bacilli from transitioning from a dormant to growth phase. Thus, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical or veterinarian.

The subject treated with the vaccine typically will have or will develop protective immunity to an infecting bacterium. The term “protective immunity” means that a vaccine, immunogenic composition or immunization schedule that is administered to a mammal induces an immune response that prevents, retards the development of, or reduces the severity of a disease that is caused by a pathogenic bacterium or diminishes or altogether eliminates the symptoms of the disease. By “infecting bacterium” is meant a bacterium that has established infection in the host, and which may be associated with a disease or undesirable symptom as a result. Generally, infecting bacteria are pathogenic bacteria.

The terms “immunogenic bacterial composition”, “immunogenic composition”, and “vaccine” are used interchangeably herein to mean a preparation capable of eliciting a cellular and/or humoral immune response in a subject when administered in a sufficient amount to elicit an immune response to epitopes present in said preparation.

A pharmaceutical composition containing the irradiated mycobacteria or brucella described above is administered to a suitable individual for preventing or treating mycobacteria or brucella. Reference herein to “tuberculosis” includes reference to pulmonary and extra-pulmonary bacilli. The terms “individual,” “subject,” “host,” and “patient,” are used interchangeably herein and refer to any subject having a bacterial infection amenable to treatment using the therapeutic vaccine of the invention, and for whom treatment or therapy is desired. The pharmaceutical composition can be prepared for any mammalian host that is susceptible to infection by mycobacteria or brucella. Suitable mammalian hosts include, e.g., farm animals such as swine and bovine

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom (c) preventing reactivation of the disease, i.e. preventing the bacilli from transitioning from a dormant to growth phase. Thus, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical or veterinarian.

The subject treated with the vaccine typically will have or will develop protective immunity to an infecting bacterium. The term “protective immunity” means that a vaccine, immunogenic composition or immunization schedule that is administered to a mammal induces an immune response that prevents, retards the development of, or reduces the severity of a disease that is caused by a pathogenic bacterium or diminishes or altogether eliminates the symptoms of the disease. By “infecting bacterium” is meant a bacterium that has established infection in the host, and which may be associated with a disease or undesirable symptom as a result. Generally, infecting bacteria are pathogenic bacteria.

The phrase “in a sufficient amount to elicit an immune response” means that there is a detectable difference between an immune response indicator measured before and after administration of a particular vaccine preparation or immunogenic composition. Several weeks after the last vaccination, animals will be challenged with live infection. The clinical and molecular immune response is evaluated several weeks after challenge with virulent bacteria.

The term “state”, “metabolic state”, “altered state”, or “different state” might be used interchangeably and refer to different metabolic states of the mycobacterium in which there is a detectable difference in protein composition or gene expression. A state is defined whereby a bacterium has responded to external stimuli and as a result the bacterium presents different antigens or may express and begin transcription of particular genes.

The term “osmotic delivery system” or “delivery system” refers to devices or compositional matrixes that allow for delivery of the inactivated bacterium to the intended immune target. An example of this delivery system might be a compositional matrix that allows for delivery of inactivated Brucella abortus into the small and large intestines of calves avoiding the degrading nature of low pH stomach.

Screening and Developing Vaccines

A test vaccine can be screened or optimized by subjecting a population of mycobacterium or bacterial cells, or fractions thereof (as described above) to various inactivation regimens, preparing a candidate pharmaceutical composition containing the treated cells or cell fractions and testing the ability of the treated composition using the methods described above to elicit an immune response and/or mount an effective challenge to mycobacterium infection in a host.

The terms “immunogenic bacterial composition”, “immunogenic composition”, and “vaccine” are used interchangeably herein to mean a preparation capable of eliciting a cellular and/or humoral immune response in a subject when administered in a sufficient amount to elicit an immune response to epitopes present in said preparation.

The terms “state” or “phase” are used interchangeably herein and refer to the mycobacterium's metabolic state in response to external stimuli or environment.

Immunopotency of the antigenic molecule expressed by the mycobacterium cell or extract preparation, can be determined by monitoring the immune response of test animals following immunization with the bacteria expressing the recombinant antigen. Test animals may include mice, guinea pigs, rabbits, bovine, non-human primates, and eventually human subjects.

The immune response of the test subject can additionally be analyzed by various approaches such as: (a) T-cell associate cytokine production (b) plasma cytokine production (c) T cell proliferation, cytoxicity, cytokine profiles (d) T cell antigen repertoire (e) T cell regulatory profiles (f) mRNA profiles (g) innate immunity profiles (h) antibody profiles (i) genetics and (j) protection from disease and/or mitigation of infectious symptoms in immunized animals.

### Composition for Preventing and Treating Asthma

In a further aspect the invention provides compositions for preventing and treating asthma using irradiated Mycobacterium species delivered directly to the respiratory system.

The majority of pathogens as well as environmental allergens invade humans though mucosal surfaces. The mucosal and pulmonary immune system has evolved site specific ways to prevent colonization and invasion by foreign antigens. Therefore, stimulation of these defenses is important for preventing infection and allergic disease.

Indirect associations between asthma and mycobacterial infection suggest a possible strategy to utilize mycobacterium to inhibit the development of allergen-induced Th2 responses and favor Th1 immune responses. An intrapulmonary or mucosally delivered approach would offer a direct localized effect and inhibit recruitment and or expansion of Th2 cells within the lungs. Gamma-Irradiated M. tb is used often in laboratories as a surrogate for live M. tb because it is highly immunogenic and safe and elicits a potent Th1 response.

In one aspect, the invention provides a method for preventing or treating asthma using a composition that includes inactivated Mycobacterium.

In another aspect, the invention provides a method for creating a pulmonary/mucosal Th1-stimulator to be used to induce immune tolerance in Th2-type allergic diseases.

Suitable Mycobacterium spp. include, e.g., M. tuberculosis, M. marinum, M. bovis, M. africanum, M. canetti, or M. microti. In some embodiments, the inactivated Mycobacterium spp. cells are killed cells or cell lysates.

In some embodiments, the Mycobacterium spp. is inactivated with irradiation. Preferably irradiation is with gamma irradiation. In other embodiments, inactivation is with ultraviolet radiation and/or xrays. In other embodiments, the Mycobacterium spp. is inactivated with formalin or heat.

In a still further aspect, the irradiated Mycobacterium is provided as a pharmaceutical composition. The pharmaceutical composition may optionally include another antigen to which an immunological response is desired and/or an adjuvant to enhance the immune response in the host. The pharmaceutical composition may in addition include a pharmaceutically acceptable carrier or be provided lyophilized. In some embodiments, the pharmaceutical composition is formulated for intranasal delivery to the host.

In some embodiments, the adjuvant may be combined with toll like receptor agonists or pattern recognition receptor agonists. Suitable toll like receptor agonists include but are not limited to, e.g., TLR2, TLR4, TLR7/8 and TLR9 agonists

In some embodiments, inactivated Mycobacterium spp. are combined with carriers such as inert microparticles or liposomes.

In some embodiments, irradiated Mycobacterium spp. are combined with aluminum salts.

In some embodiments, inactivated spp. are combined with water in oil or oil in water emulsions.

In addition, the pharmaceutical composition is provided as an aerosol, spray package, or delivered by a pressurized cartridge.

In one embodiment, the invention provides a pharmaceutical composition that includes a gamma-irradiated Mycobacterium spp. that is formulated for intranasal or intrapulmonary delivery to a mammalian host and confers an immune-stimulating dose when delivered to the host, e.g., a human.

In another aspect, the invention provides a method of acting as an immunostimulant or immunomodulator against allergy induced asthma. The method includes administering to the mammal a composition comprising inactivated Mycobacterium spp., wherein the vaccination of the mammal is intranasal or intrapulmonary and wherein the composition comprises an immunologically stimulating dose when delivered to the host.

In another aspect, the invention may act as an immune-stimulant and be combined with other agents such as proteins, pharmaceutical preparations, antigens, therapeutic agents, virus-like particles, and other bacterial components.

In some embodiments, the Mycobacterium spp. for use in the method is inactivated with irradiation. Preferably irradiation is with gamma irradiation. In other embodiments, the Mycobacterium spp. is inactivated with formalin, heat, or osmotic pressure.

The pharmaceutical composition for use in the method may optionally include an additional adjuvant to further enhance an immune response in the host.

The pharmaceutical composition for use in the method may optionally include a pharmaceutically acceptable carrier or be provided lyophilized.

In some embodiments, the pharmaceutical composition for use in the method is formulated for intranasal delivery to the host.

In addition, the pharmaceutical composition for use in the method is provided as an aerosol or spray package.

In some embodiments the pharmaceutical composition is delivered through a device configured for mucosal, nasal or pulmonary delivery.

In a still further aspect, the invention provides a method for preparing a vaccine for treating Mycobacterium infection, comprising formulating an immunologically stimulating dose of an inactivated Mycobacterium spp. for intranasal or pulmonary delivery to a mammalian host.

In some embodiments, the method include for testing the adjuvant or protective asthma dose in a non-human animal model of tuberculosis. The animal model can be, e.g., a mouse, guinea pig, rabbit, bovine, or non-human primate.

In some embodiments, the inactivated mycobacterium may be combined with other adjuvants including inorganic salts, oligonucleotides, oil emulsions and saponin based mixtures.

In some embodiments, the inactivated mycobacterium may be combined with cholera toxin (CT), E. coli heat labile toxin (LT), CpG oligonucleotides, DNA, or microparticulates such as virosomes, liposomes, cochleates, polymeric microspheres, mucoadhesive polymers, or immunostimulating complexes (ISCOMs).

In some embodiments, the inactivated mycobacterium may be combined with lipid-based adjuvant or delivery system. These include but are not limited to liposomes (anionic and cationic closed vesicles made from ester lipids), proteoliposomes, cochleates (liposomes converted to rolled-up bilayer sheets with no aqueous spaces) and proteoliposome cochleates, Iscomatrix, virosomes, Eurocine, and monophosphoryl lipid A.

In some embodiments, the irradiated Mycobacterium is combined with bacterial pathogens or their components. Bacteria include, but are not limited to, Streptococcus pneumonia, Neisseria meningitides, Group A streptococci, Group B streptococci, Staphylococcus aureus, Staphylococcus epidermidis, Enterococcus faecalis, Pseudomonas aeruginosa, Escherichia coli, Klebsiella pneumonia, Chlamydia trachomatis, and Helicobacter pylori.

In some embodiments, the irradiated Mycobacterium is combined with attenuated or inactivated viral components or whole viruses. Their virus or combination of viruses that are common causes of respiratory infections. Human viruses include rhinovirus, coronavirus, influenza, human parainfluenza viruses, human respiratory syncytial virus, adenovirus, enterovirus, Paramyxoviruses and metapneumovirus. The composition may include coxsackie, echo, herpes simplex virus, corona, epstein bar virus and/or cytomegalo virus.

In some embodiments, the irradiated Mycobacterium may be used in conjunction with viruses that may cause disease in livestock such as viruses: PI3, IBR, BVD, BRSV, Adenovirus, Rhinovirus, Herpesvirus IV, Enterovirus, MCF and Reovirus.

In some embodiments, the proposed irradiated Mycobacterium may be used in conjunction with suspected pathogens that cause bovine calicivirus, bovine parvovirus, BHV4, bovine reovirus, bovine enterovirus, bovine rhinovirus, and malignant catarrhal fever virus.

In other embodiments, the proposed adjuvant may be used in conjunction with bovine rhinotracheitis virus, a type 1 bovine herpesvirus, parainfluenza virus type 3, bovine respiratory syncytial virus, bovine viral diarrhea virus, bovine adenovirus and bovine coronavirus.

In some embodiments, as deemed appropriate by a person skilled in the art, these viruses or their components may be delivered via vectors such as DNA vectors. In other embodiments, the proposed therapeutic may be used in conjunction with killed bacteria such as Hemophilus, Ureaplasma diversum, Mycoplasma dispar, Mycoplasma bovis, and Mycoplasma bovirhinis, Pasteurellaceae, including Mannheimia haemolytica, Pasteurella multocida, Haemophilus somnus,

A composition for treating asthma according the invention is prepared using one or more inactivated Mycobacterium spp. that is then formulated for pulmonary and mucosal delivery to a subject. Methods for preparing the inactivated Mycobacterium spp. are described in WO2008/128065 and US20100112007. It is widely known and excepted by a person skilled in the art that mycobacterium can be inactivated using gamma irradiation and that the dose necessary to kill Mycobacterium tuberculosis to a 10²⁰ degree of certainty is 2.4 megarads. For example, cells can be irradiated using 137Cs (Cesium) for 27 hours at 1543 rads/minute for a total dosage of 2.5 megarads.

The inactivated Mycobacterium, when delivered to the lung, orally or to the mucosa of a subject, is postulated to act as an immunomodulator for prophylaxis against allergy-induced asthma and as an immunostimulant or adjuvant to stimulate cellular immune responses.

While not wishing to be bound by theory, the inventor surmises that the immune response elicited from aerosolized inactivated Mycobacterium spp. when delivered to the respiratory mucosa may offer a unique ability to evoke an immune response in the pulmonary and respiratory system by direct stimulation of antigen presenting cells in the respiratory epithelium. Over 90% of infectious diseases as well as environmental allergens invade the host though mucosal surfaces and stimulation of mucosal immunity may be the best approach to control such infections and allergens^{[68,69]}. The ideal route for an asthma vaccine should be selected based on the site of allergen invasion, the respiratory system, where there exists some compartmentalization.

Recent evidence suggests that pulmonary lymphocytes remain localized when mounting an initial immune response and only a limited number of B and T-cells migrate systemically^{[34,35]}. The human pulmonary lymph anatomy is unique in that cells entering the thoracic duct from the local pulmonary nodes travel back to the lung in the pulmonary arterial blood before reaching other tissues. Some lymphocytes may pass through to the systemic circulation, but activated T-cells tend to adhere to the vascular endothelium and move back into the lung, thus keeping the T-cells near the foci of infection^{[36]}. Therefore, targeting the airway luminal and mucosal immune cells holds important implications for developing effective vaccination strategies.

Several studies have found inverse relationships between TST reactions and asthma or/and atopy^{[70,71,72,73]}. The International Study of Asthma and Allergies in Childhood, an ecological study in over 700,000 children found children ages 6-7 to be significantly (p<0.0001) less likely to have wheezing in regions where TB notification rates and WHO TB incidence rates were high^{[74,75]}. Given these indirect associations between mycobacterial infection and asthma, several researchers found prophylaxis treatment with both live and heat killed BCG in mice lead to polarized Th1 immune responses in the lung and inhibited the development of allergen-induced Th2 responses^{[76,77,78,79,80]}. Specifically, live or dead mycobacteria inhibited recruitment and/or expansion of Th2 cells homing into the lung, increased IFN-γ levels and decreased the eosinophilia after ovalbumin airway challenge^{[76,77,78,79,80]}.

Given such observations, a recent pilot study investigated the effect of intranasal delipidated acid-treated M. vaccae on adults with asthma and found no significant difference between the treatment group and placebo^{[81]}. ^{[81]}These results may not be surprising, for timing of microbe or allergen co-exposure appears to be relevant. As illustrated in the murine model of allergic airway disease, transfer of allergen specific Regulatory T-cells prevented, though did not reverse, airway remodeling changes in a chronic challenge model^{[82,83]}, possibly illustrating that a developing immune system may hold greater potential for abating asthmatic inflammation.

Shirtcliffe et. al^{[84]} investigated the effect of repeated intradermal injections of heat-inactivated Mycobacterium bovis bacillus Calmette-Guérin in adult asthma. The recruitment to the trial was halted early and the number of injections reduced in a number of patients due to excessive local reactions to BCG. The lack of efficacy of repeated heat-inactivated BCG injections together with the adverse reactions limits the therapeutic potential of heat killed BCG. However, the process of heat killing mycobacterium has been shown to denature proteins and enzymes which may both contribute to adverse reactions and lack of efficacy. Moreover, the systemic approach of providing the vaccination may further reduce the potential efficacy of intradermal heat-killed BCG. Gamma irradiation has been shown to help preserve protein structure while fully inactivating the bacilli. Therefore, the aforementioned proposal to deliver aerosolized gamma irradiated mycobacterium may confer unique advantages both in the localized delivery and in the antigenic presentation.

Aerosolized or mucosally delivered irradiated Mycobacterim may be used to treat various types of asthma-related conditions, including bronchial, allergic, intrinsic, extrinsic, exercise-induced, drug-induced (including aspirin and NS AID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitis and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.

It is postulated that the idea of using whole dead mycobacteria such as irradiated M.tb as a mucosal/pulmonary immunostimulant has been overlooked due to fear of hyper-inflammation in an individual previously infected with M. tb, in what is known as the Koch-phenomenon. However, clinical use of mycobacteria as a therapeutic over the past several decades provides compelling evidence regarding its safety. Hundreds of thousands of individuals have received high intravesicular doses (10⁸)×6 doses of live BCG, and there is no report in the medical literature of any Koch-like reaction. Moreover, one pioneering study performed in 1968 on hundreds of children and college students delivered aerogenic live BCG, also at high doses, and the researchers noted no respiratory dysfunction or fever in any of the participants^{[48]}. Finally, intradermal vaccination with killed M. vaccae entered trials a couple of years ago, for evaluation as a TB vaccine and asthma therapeutic. Though efficacy for each study proved to be minimal, no Koch-like reaction was reported in the thousands of individuals receiving the M. vaccae^{[81,85,86,87,88,89]}.

The invention provides for methods for eliciting an immune response to asthma which may include administering an effective amount of any one of the immunogenic or vaccine compositions of the present invention to induce the response in a mammalian subject, e.g., a human or bovine. The invention also provides for methods for inducing an immunological or protective response which may comprise administering an effective amount of any one of the immunogenic or vaccine compositions of the present invention to induce the response in a desired mammalian subject.

The invention also encompasses method of stimulating acquisition of protective immunity which may comprise administering an effective amount of inactivated Mycobacterium spp. prior to vaccination with an effective amount of a vaccine. The invention also provides for kits encompassing the compositions and/or methods described herein.

The inactivated Mycobacterium app. can be used to decrease an allergic Th2 response to an antigen. “Antigen” is herein defined as a compound which, when introduced into an animal or a human, will result in allergic symptoms.

“Vaccine” is herein defined as a composition of antigenic moieties, usually consisting of modified-live (attenuated) or inactivated infectious agents, or some part of the infectious agents, that is administered into the body to produce active immunity.

Animal asthma models known in the art can be used to characterize the response to IrrM.Tb. One model is the well-characterized murine model of allergic asthma, describe in U.S. Pat. No. 7,553,487, in which allergen exposure leads to airway hyper responsiveness (“AHR”), pulmonary eosinophilia, elevations in antigen-specific serum IgE levels, and increases in airway epithelial mucus content. The asthma response can additionally be analyzed using known allergic effector cascades. Eosinophils have been implicated as primary effector cells in asthma and asthmatic AHR. Allergic asthma in murine models is associated with a marked increase in the mucus content of the airway epithelium. Mucus hypersecretion is particularly profound in autopsy specimens from patients who die of acute asthma attacks.

The irradiated Mycobacterium described herein can addition be used to create positive and negative controls for immune responses whereas, the formulation is useful as a benchmark to compare the immune responses of other agents.

### Composition and Method for Enhancing an Immune Response

In a further aspect, the invention provides an irradiated Mycobacterium species as an adjuvant to stimulate cellular immune responses.

The majority of pathogens such as viruses, bacteria and parasites, as well as environmental allergens, invade humans though mucosal surfaces. The mucosal and pulmonary immune system has evolved site specific ways to prevent colonization and invasion by harmful pathogens. Therefore, stimulation of these defenses is important for preventing infection and controlling disease. Adjuvants can trigger early innate immune responses involved in the creation of strong, protective immune responses and are critical to a vaccine's efficacy. Thus, the role of immunostimulants is becoming more important in vaccinology.

Gamma-Irradiated M. tb (IrrM.tb) is used often in laboratories as a surrogate for live M. tb because it is highly immunogenic and safe. IrrM.tb examination into its use as a T helper-1 immunostimulator has been overlooked. An aerosolized or mucosal approach would afford a localized immune response. It may offer advantages since the approach would mimic antigen invasion and that there may be a degree of compartmentalization within the respiratory system. Since there is currently no FDA-approved mucosal adjuvant or aerosolized immunostimulants, new strategies are needed for designing and developing mucosal and inhaled vaccines and therapeutics.

In one aspect the invention provides a method for creating an adjuvant or immunostimulant using irradiated Mycobacterium for delivery to a subject without apparent symptoms or other indicia of tuberculosis but for whom enhancement of an immune response is desired.

In another aspect, the invention provides a method for creating a pulmonary/mucosal Th1-stimulation to be used as an adjuvant for vaccines or therapeutics.

Suitable Mycobacterium spp. include, e.g., M. tuberculosis, M. marinum, M. bovis, M. africanum, M. canetti, or M. microti. In some embodiments, the inactivated Mycobacterium spp. cells are killed cells or cell lysates.

In some embodiments, the Mycobacterium spp. is inactivated with irradiation. Preferably irradiation is with gamma irradiation. In other embodiments, inactivation is with ultraviolet radiation and/or xrays. In other embodiments, the Mycobacterium spp. is inactivated with formalin or heat.

In a still further aspect, the irradiated Mycobacterium is provided as a pharmaceutical composition. The pharmaceutical composition may optionally include another antigen to which an immunological response is desired and/or an adjuvant to enhance the immune response in the host. The pharmaceutical composition may in addition include a pharmaceutically acceptable carrier or be provided lyophilized. In some embodiments, the pharmaceutical composition is formulated for intranasal delivery to the host.

In some embodiments, the adjuvant may be combined with toll like receptor agonists or pattern recognition receptor agonists. Suitable toll like receptor agonists include but are not limited to, e.g., TLR2, TLR4, TLR7/8 and TLR9 agonists

In some embodiments, irradiated Mycobacterium spp. are combined with carriers such as inert microparticles or liposomes.

In some embodiments, irradiated Mycobacterium spp. are combined with aluminum salts.

In some embodiments, irradiated Mycobacterium are combined with water in oil or oil in water emulsions.

In addition, the pharmaceutical composition is provided as an aerosol, spray package, or delivered by a pressurized cartridge.

In one embodiment, the invention provides a pharmaceutical composition that includes a gamma-irradiated Mycobacterium spp. that is formulated for intranasal or intrapulmonary delivery to a mammalian host and confers an immune-stimulating dose when delivered to the host, e.g., a human.

In another aspect, the invention may act as an immune-stimulant and be combined with other agents such as proteins, pharmaceutical preparations, antigens, therapeutic agents, virus-like particles, and other bacterial components.

In some embodiments, the Mycobacterium for use in the method is inactivated with irradiation. Preferably irradiation is with gamma irradiation. In other embodiments, the Mycobacterium spp. is inactivated with formalin, heat, or osmotic pressure.

The pharmaceutical composition for use in the method may optionally include an additional adjuvant to further enhance an immune response in the host.

The pharmaceutical composition for use in the method may optionally include a pharmaceutically acceptable carrier or be provided lyophilized.

In some embodiments, the pharmaceutical composition for use in the method is formulated for intranasal delivery to the host.

In addition, the pharmaceutical composition for use in the method is provided as an aerosol or spray package.

In some embodiments the pharmaceutical composition is delivered through a device configured for mucosal, nasal or pulmonary delivery.

In a still further aspect, the invention provides a method for preparing a vaccine for treating Mycobacterium infection, comprising formulating an immunologically stimulating dose of an inactivated Mycobacterium spp. for intranasal or pulmonary delivery to a mammalian host.

In some embodiments, the method include for testing the adjuvant dose in a non-human animal model of tuberculosis. The animal model can be, e.g., a mouse, guinea pig, rabbit, bovine, or non-human primate.

In some embodiments, the inactivated mycobacterium may be combined with other adjuvants including inorganic salts, oligonucleotides, oil emulsions and saponin based mixtures.

In some embodiments, the inactivated mycobacterium may be combined with cholera toxin (CT), E. coli heat labile toxin (LT), CpG oligonucleotides, DNA, or microparticulates such as virosomes, liposomes, cochleates, polymeric microspheres, mucoadhesive polymers, or immunostimulating complexes (ISCOMs).

In some embodiments, the inactivated mycobacterium may be combined with lipid-based adjuvant or delivery system. These include but are not limited to liposomes (anionic and cationic closed vesicles made from ester lipids), proteoliposomes, cochleates (liposomes converted to rolled-up bilayer sheets with no aqueous spaces) and proteoliposome cochleates, Iscomatrix, virosomes, Eurocine, and monophosphoryl lipid A.

In some embodiments, the aforementioned adjuvant is combined with bacterial pathogens or their components. Bacteria include, but are not limited to, Streptococcus pneumonia, Neisseria meningitides, Group A streptococci, Group B streptococci, Staphylococcus aureus, Staphylococcus epidermidis, Enterococcus faecalis, Pseudomonas aeruginosa, Escherichia coli, Klebsiella pneumonia, Mycobacterium tuberculosis, Chlamydia trachomatis, and Helicobacter pylori.

In some embodiments, the aforementioned adjuvant is combined with attenuated or inactivated viral components or whole viruses. Human viruses include rhinoviruses, coronaviruses, influenza viruses, adenovirues, human parainfluenza viruses, human respiratory syncytial viruses, adenoviruses, enteroviruses, Paramyxoviruses and metapneumoviruses. The composition may include viruses in the genera coxsackie, echovirus, ebstein bar virus and cytomegalo virus.

In some embodiments, the proposed adjuvant may be used in conjunction with viruses that may cause disease in livestock such as viruses: PI3, IBR, BVD, BRSV, Adenovirus, Rhinovirus, Herpesvirus IV, Enterovirus, MCF and Reovirus.

In some embodiments, the proposed adjuvant may be used in conjunction with suspected pathogens that cause bovine calicivirus, bovine parvovirus, BHV4, bovine reovirus, bovine enterovirus, bovine rhinovirus, and malignant catarrhal fever virus.

In other embodiments, the proposed adjuvant may be used in conjunction with bovine rhinotracheitis virus, a type 1 bovine herpesvirus, parainfluenza virus type 3, bovine respiratory syncytial virus, bovine viral diarrhea virus, bovine adenovirus and bovine coronavirus.

In some embodiments, as deemed appropriate by a person skilled in the art, these viruses or their components may be delivered via vectors such as DNA vectors. In other embodiments, the proposed adjuvant may be used in conjunction with bacteria such as Hemophilus, Ureaplasma diversum, Mycoplasma dispar, Mycoplasma bovis, and Mycoplasma bovirhinis, Pasteurellaceae, including Mannheimia haemolytica, Pasteurella multocida, Haemophilus somnus.

A composition according the invention is prepared using one or more inactivated Mycobacterium spp. that is then formulated for pulmonary and mucosal delivery to a subject. Methods for preparing the inactivated Mycobacterium spp. are described in WO2008/128065 and US20100112007. Mycobacterium can be inactivated using gamma irradiation and it is understood that the dose necessary to kill Mycobacterium tuberculosis to a 10²⁰ degree of certainty is 2.4 megarads. For example, cells can be irradiated using 137Cs (Cesium) for 27 hours at 1543 rads/minute for a total dosage of 2.5 megarads.

The inactivated Mycobacterium, when delivered to the lung, orally, or to the mucosa of a subject is postulated to act as an immunomodulator or adjuvant to stimulate cellular immune responses. The inactivated Mycobacterium can be delivered to the mucosal or intrapulmonary system to act as a vaccine adjuvant or therapeutic to activate T cell subsets (Th1, Th17, Regulatory T cells). Irradiated Mycobacterium has specific characteristics making it an intriguing compound as an immunostimulant. Antigens on the cell wall of irrM.tb elicit innate immune responses similar to live M. tb^{[25,26,28,90]} Gamma-irradiated mycobacterium undergoes apoptosis and can become engulfed by dendritic cells (DC). DC present the mycobacterium antigens to T-cells, which activate CD4 Th1 and CD8 cytotoxic cells^{[}26]. Gamma-irradiated M tb can also induce nitric oxide release^{[25]} and can elicit similar T-helper (Th) responses to live M tb^{[26]}.

While not wishing to be bound by theory, the inventor surmises that the immune response elicited from aerosolized inactivated Mycobacterium spp. when delivered to the respiratory mucosa may offer a unique ability to evoke an immune response in the pulmonary and respiratory system by direct stimulation of antigen presenting cells in the respiratory epithelium. Over 90% of infectious diseases as well as environmental allergens invade the host though mucosal surfaces and stimulation of mucosal immunity may be the best approach to control such infections and allergens^{[68]}. Most of the current vaccines are delivered systemically and these fail to elicit respiratory or mucosal immunity.^{[68]}. The ideal immunization route should be selected based on the site of pathogen invasion and in the respiratory system there exists a degree of compartmentalization.

Recent evidence suggests that pulmonary lymphocytes remain localized when mounting an initial immune response and only a limited number of B and T-cells migrate systemically^{[34,35]}. Compared to other mucosal tissues, the lung vasculature and interstitum trap more circulating T cells^{[91]}. The human pulmonary lymph anatomy is unique in that cells entering the thoracic duct from the local pulmonary nodes travel back to the lung in the pulmonary arterial blood before reaching other tissues. Some lymphocytes may pass through to the systemic circulation, but activated T-cells tend to adhere to the vascular endothelium and move back into the lung, thus keeping the T-cells near the foci of infection^{[36]}. Therefore, targeting the airway luminal and mucosal immune cells holds important implications for developing effective vaccination strategies. Additionally, airway luminal/mucosally delivered vaccines would have significant advantages such as eliminating the need of needles and enabling rapid vaccination responses in the face of pandemics. As such is vast commercial potential for airway/luminal vaccinations.

Mycobacteria possess inherent adjuvant properties. These properties include ability to activate dendritic cells (DC) through Toll-like receptors (TLRs)^{[92][93,94,95]}, induce proinflammatory cytokines and target intracellular MHC-processing compartments. Experimental studies evaluating respiratory mucosal adjuvants have largely been dedicated to the method of intranasal delivery or oral mucosal delivery but this may differ from aerosol delivery of vaccines in terms of anatomical induction of immune responses. Aerosol vaccination by inhalation may effectively target the small airway. In this regard, dried BCG delivered from an inhaler to guinea pigs was shown to significantly reduce bacilli burden^{[96]}. The effect of mucosal and airway luminal vaccination by aerosolization still remains to be evaluated.

It is postulated that the idea of using whole dead mycobacteria such as irradiated Mycobacterial species as a mucosal/pulmonary immunostimulant has been overlooked due to fear of hyper-inflammation in an individual previously infected with M. tb, in what is known as the Koch-phenomenon. However, clinical use of mycobacteria as a therapeutic over the past several decades provides compelling evidence regarding its safety. Hundreds of thousands of individuals have received high intravesicular doses (10⁸)×6 doses of live BCG, and there is no report in the medical literature of any Koch-like reaction. Moreover, one pioneering study performed in 1968 on hundreds of children and college students delivered aerogenic live BCG, also at high doses, and the researchers noted no respiratory dysfunction or fever in any of the participants^{[48]}. Finally, intradermal vaccination with killed M. vaccae entered trials a couple of years ago, for evaluation as a TB vaccine and asthma therapeutic. Though efficacy for each study proved to be minimal, no Koch-like reaction was reported in the thousands of individuals receiving the M. vaccae^{[81,85,86,87,88,89]}.

The invention provides for methods for eliciting an immune response which may comprise administering an effective amount of any one of the immunogenic or vaccine compositions of the present invention to induce the response in a mammalian subject, e.g., a human or bovine. The invention also provides for methods for inducing an immunological or protective response which may comprise administering an effective amount of any one of the immunogenic or vaccine compositions of the present invention to induce the response in a desired mammalian subject.

The invention also encompasses method of stimulating acquisition of protective immunity which may comprise administering an effective amount of inactivated Mycobacterium spp. prior to vaccination with an effective amount of a vaccine. The invention also provides kits encompassing the compositions and/or methods described herein.

The inactivated Mycobacterium app. can be used to increase the response to an antigen or vaccine. “Antigen” is herein defined as a compound which, when introduced into an animal or a human, will result in the formation of antibodies and cell-mediated immunity.

“Adjuvant” is herein defined as a compound or compounds that, when used in combination with specific vaccine antigens in formulations, augment or otherwise alter or modify the resultant immune responses.

“Vaccine” is herein defined as a composition of antigenic moieties, usually consisting of modified-live (attenuated) or inactivated infectious agents, or some part of the infectious agents, that is administered, most often with an adjuvant, into the body to produce active immunity.

The antigen for use in may be any desired antigen falling within the definition set forth above. Antigens are commercially available or one of skill in the art is capable of producing them. The antigenic moiety making up the vaccine can be either a modified-live or killed microorganism, or a natural product purified from a microorganism or other cell including, but not limited to, tumor cells, a synthetic product, a genetically engineered protein, peptide, polysaccharide or similar product, or an allergen. The antigenic moiety can also be a subunit of a protein, peptide, or polysaccharide. The antigen may also be the genetic antigens, i.e., the DNA or RNA that engenders an immune response. Representative of the antigens that can be used according to the present invention include, but are not limited to, natural, recombinant or synthetic products derived from viruses, bacteria, fungi, parasites and other infectious agents in addition to autoimmune diseases, hormones, or tumor antigens which might be used in prophylactic or therapeutic vaccines and allergens. The viral or bacterial products can be components which the organism produced by enzymatic cleavage or can be components of the organism that were produced by recombinant DNA techniques that are well known to those of ordinary skill in the art. Because of the nature of the invention and its mode of delivery it is very conceivable that the invention would also function as a delivery system for drugs, such as hormones, antibiotics and antivirals.

The irradiated Mtb described herein can addition be used to create positive and negative controls for immune responses, e.g., in a formulation useful as a benchmark to compare the immune responses of other agents.

Streptococcus pyogenes Composition for Mucosal Delivery and Method of Using Same

The invention additionally provides a composition for enhancing an immune response and more particularly to a composition of a Streptococcus pyogenes formulated for mucosal delivery. Preferably, inactivated S. pyogenes are used to procure an immune response and provide protection against future S. pyogenes exposure to reduce invasive and non invasive infection.

Streptococcus pyogenes, also known as Group A streptococcus is a member of the group β-hemolytic streptococci. S. pyogenes is considered the most common pathogenic bacteria that infect children and adolescents and thus is a considerable health problem. While most strains of S. pyogenes cause infections that are transient and relatively innocuous, some strains can cause significant morbidity. S. pyogenes is a producer of noninvasive disease, such as as pharyngitis, otitis media, and subsequent acute rheumatic fever and acute glomerulonephritis. Invasive infections caused by group A Streptococcus include necrotizing fasciitis (NF), bacteremic pneumonia, sepsis, and streptococcal toxic shock syndrome. S. pyogenes commonly presents as pharyngitis in ages of 5-15 years and is thought to be responsible for as high as 30% of childhood pharyngitis^{[97]}. Moreover, it is estimated that group A Streptococcus is responsible for 500,000 deaths annually and financial costs are estimated to be about $500 million annually within the United States alone.^{[98]} Since approximately 91% of invasive S. pyogenes cases are hospitalized, there is a significant burden on the healthcare system that has yet to be addressed^{[99]}

The invention provides pharmaceutical composition that includes one or more types S. pyogenes, preferably formulated for mucosal delivery to a subject. A preferred method of inactivating S. pyogenes is via gamma irradiation. The pharmaceutical compositions may be used with an adjuvant or used in conjunction with attenuated, non-infectious, or inactivated bacteria or its cell lysates.

In one aspect, the composition is used as a vaccine against Streptococcus pyogenes.

In one aspect, the composition provides a therapeutic for non invasive and invasive disease.

The proposed composition of inactivated bacteria can be utilized with a number of vaccination strategies: prophylactically-given prior to infection to prevent infection with bacteria, post-exposure to eliminate or contain latent and prevent reactivation. It can either be used to replace a current vaccine and/or as a booster to other vaccines in patients who have already the appropriate vaccination.

In one aspect, the invention provides a pharmaceutical composition comprising gamma irradiated S. pyogenes, wherein the composition is formulated for intranasal, mucosal or intrapulmonary delivery to a mammalian host, and wherein the composition comprises an immunologically protective dose when delivered to the host.

In one aspect, the composition includes a population of Streptococcus in a defined state. A state of Streptococcus can mean, e.g., cells in states arising from nutritional deprivation, extreme temperatures, iron depletion, aerobic growth, anaerobic growth, oxidative stress, or a combination of two or more of these states. In some embodiments, more than 90%, 95%, 98%, 99% or 99.9% of the cells are in the predetermined state.

In some embodiments, the inactivated S. pyogenes cells are killed cells or cell lysates.

In general, many bacterial species or strain can be used in the composition and methods of the invention. In one aspect, the invention provides of pharmaceutical composition of one or more different states of bacteria spp, wherein the states may be a result of exposing the bacteria prior to inactivation to various stimuli.

In another aspect, the bacteria are exposed to different stimuli or environments to allow for different antigenic expression.

In some embodiments, some of the bacteria are inactivated or attenuated.

In some embodiments, the bacteria is inactivated with irradiation. Preferably irradiation is with gamma irradiation but other types of radiation may be used including xray and microwaves.

In some embodiments, the bacteria is inactivated with osmotic pressure via salts or drying process.

The pharmaceutical composition may optionally include an adjuvant to enhance an immune response in the host.

In some embodiments, S. pyogenes is combined with the inactivated Mycobacterium spp., the latter of which may act as an adjuvant.

The pharmaceutical composition may optionally include a pharmaceutically acceptable carrier, or be provided lyophilized.

The pharmaceutical composition may optionally include a pharmaceutically acceptable carrier such as glucose, lactose or sorbitol.

In some embodiments, the pharmaceutical composition is formulated for intranasal delivery to the host.

In addition, the pharmaceutical composition is provided as an aerosol or spray package.

In one embodiment, the invention provides a pharmaceutical composition that includes a gamma-irradiated S. pyogenes spp. that is formulated for intranasal or intrapulmonary delivery to a mammalian host and which confers an immunologically protective dose when delivered to the host, e.g., a human.

In another aspect, the invention provides a method of vaccinating a mammal against infections caused by S. pyogenes. The method includes administering to the mammal a composition comprising inactivated S. pyogenes spp., wherein the vaccination of the mammal is intranasal or intrapulmonary, and wherein the composition comprises an immunologically protective dose when delivered to the host.

In another aspect, the invention provides an immunostimulant that facilitates delivery of another antigen.

In one aspect, the invention provides a pharmaceutical composition comprising an inactivated S. pyogenes spp., wherein the composition is formulated for intranasal, mucosal or intrapulmonary delivery to a mammalian host, and wherein the composition comprises an immunologically protective dose when delivered to the host.

In some embodiments, the inactivated cells are killed cells or cell lysates. When the subject is a human, 100% of the S. pyogenes spp. cells are preferably inactivated.

In some embodiments, the inactivated cells are mixed with attenuated strains of bacteria.

Preferably irradiation is with gamma irradiation.

The pharmaceutical composition for use in the method may optionally include an adjuvant to enhance a protective immune response in the host.

The pharmaceutical composition for use in the method may optionally include a pharmaceutically acceptable carrier, or be provided lyophilized.

In some embodiments, the pharmaceutical composition for use in the method is formulated for intranasal delivery to the host.

In addition, the pharmaceutical composition for use in the method is provided as an aerosol or spray package.

In some embodiments, the pharmaceutical composition is delivered through a device configured for nasal or pulmonary delivery.

In some embodiments, the pharmaceutical composition is delivered via a chewable capsule, lozenge, dissolvable film or gum.

In a still further aspect, the invention provides a method for preparing a vaccine for treating S. pyogenes infection, comprising formulating an immunologically protective dose of irradiated S. pyogenes for intranasal or pulmonary delivery to a mammalian host.

In some embodiments, the method includes testing the vaccine in a non-human animal model. The animal model can be, e.g., a mouse, guinea pig, rabbit, bovine, or non-human primate.

In one embodiment, the invention includes the use of different sugars as fine and coarse carriers as part of the composition for gamma irradiated S. pyogenes. The mucosally delivered S. pyogenes can be used as part of a composition containing bacteria or viral components, either as whole entities or as partial components. The localized delivery of S. pyogenes to the mucosal surfaces may provide a suitable vaccine against both diseases such as pharyngitis, skin and soft tissue infections such as necrotizing fasciitis or bacteremic pneumonia and poststreptococcal diseases such as glomerulonephritis or rheumatic fever.

Streptococcus pyogenes, also known as Group A streptococcus is a member of the group β-hemolytic streptococci. S. pyogenes is considered the most common pathogenic bacteria that infect children and adolescents and thus is a considerable health problem. While most strains of S. pyogenes cause infections that are transient and relatively innocuous, some strains can be deadly. S. pyogenes is a producer of noninvasive disease, such as as pharyngitis, otitis media, and post-infectious diseases such as acute rheumatic fever and acute glomerulonephritis. Invasive infections caused by group A Streptococcus include necrotizing fasciitis, bacteremic pneumonia, sepsis, and streptococcal toxic shock syndrome. Resource poor regions typically are affected by acute rheumatic fever, invasive disease, rheumatic heart disease, glomerulonephritis post acute streptococcal infection, and endemic streptococcal impetigo. Resource rich countries face pharyngitis and invasive disease as great public health priorities.^{[100]}

S. pyogenes commonly presents as pharygitis in ages 5-15 years and is thought to be responsible for as high as 30% of childhood pharyngitis^{[97]}. Moreover, it is estimated that group A Streptococcus is responsible for 500,000 deaths annually and costs approximately $500 million annually within the United States alone.^{[98]} Since approximately 91% of invasive S. pyogenes cases are hospitalized, there is a significant burden on the healthcare system that has yet to be addressed^{[99]}

Research has shown that the emm gene of Streptococcus pyogenes encodes the cell surface M virulence protein for at least 100 known M sero-specificities of S. pyogenes^{[101]}. According to a review of studies from 1990-2009 that describe the epidemiology of Group A strep based on emm or M typing, a total of 205 emm types have been identified.^{[100]}. While regional and clinical manifestation may be different, the most common S. pyogenes emm types are typically emm1, emm12, emm28, emm3, and emm4. According to data of 2004 from Centers for Disease Control and Prevention's Active Bacterial Core surveillance, the 30 most common emm types accounted for 95% of isolates whereas emm Types 1 (22%), 3 (9%), 28 (9%), 12 (9%), and 89 (6%) were the most common and cumulatively accounted for 55% of isolates. Moreover, the 26 most common emm types accounted for 93% of isolates. The proportion of disease accounted for by emm types varied little over 10 years of surveillance and was similar among young children and the elderly.^{[99]}

It is envisioned that mucosal delivery of inactivated S. pyogenes will help procure an immune response and provide protection against future S. pyogenes exposure to help reduce invasive and non invasive infection. In one embodiment a portfolio of inactivated emm types are used to provide maximum protection. In another embodiment, the invention is prepared using one or more emm types of S. pyogenes that are then formulated for mucosal delivery to a subject. The composition, when delivered to mucosal/nasal mucosa of a subject is postulated to elicit a localized immune response.

One reason the inventors believe the use and formulation of inactivated mucosally delivered S. pyogenes has been overlooked is that the proposed invention relies on the inventor's unique and proprietary insights with irradiated mycobacterium tuberculosis. Thus, the need was not apparent or readily assumed. The inventors proposed in an earlier invention to aerosolize irradiated mycobacterium tuberculosis as a means to promote immunity and have unpublished data to support its use. As exposure to inactivated S. pyogenes may promote further antigen presentation of the macrophage, the inventors hypothesize that the potential vaccine will foster a long term immune response. Moreover, the route of administration follows that S. pyogenes delivered to the mucosa may offer additional roles as an immunomodulator, therapeutic agent, or adjuvant.

The bacteria to be used in the pharmaceutical composition can include whole cells or portions of cells, e.g., cell lysates. For example, suitable components include a gamma irradiated whole cell lysate, gamma irradiated culture filtrate proteins, gamma irradiated cell wall fraction, gamma irradiated cell membrane fraction, gamma irradiated cytosol fraction, gamma irradiated soluble cell wall proteins, and gamma irradiated soluble protein pool.

The bacteria to be used in the pharmaceutical composition can also include attenuated strain together with inactivated S. pyogenes.

Preparing Pharmaceutical Compositions

Inactivated S. pyogenes is prepared for administration to a host by combining with a pharmaceutically acceptable carrier to form a pharmaceutical composition. The carrier can be glucose, sucrose, lactose, sorbitol, e.g., such as physiological saline, mineral oil, vegetable oils, aqueous sodium carboxymethyl cellulose, or aqueous polyvinylpyrrolidone. Methods can be performed as described in, e.g., WO/2008/128065, or its US national phase counterpart application 20100112007, the contents of which are incorporated by reference in their entirety. Inactivated S. pyogenes is prepared for administration to a host by combining inactivated cells or cell lysates with a pharmaceutically acceptable carrier to form a pharmaceutical composition. The carrier can be glucose, sucrose, lactose, sorbitol, e.g., such as physiological saline, mineral oil, vegetable oils, aqueous sodium carboxymethyl cellulose, or aqueous polyvinylpyrrolidone. In some embodiments, the carrier is sufficiently pure to be administered therapeutically to a human subject. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants and/or other additives may be included, as required. A skilled person in the field familiar with the protocols, formulations, dosages and clinical practice associated with, e.g., the administration of S. pyogenes can in addition readily adapt these protocols for use with pharmaceutical compositions of the present invention. The immunomodulating form of S. pyogenes is administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are dependent on etiology, use, dose, and condition of the host. Suitable regimens for initial administration and booster shots are also variable but are typified by an initial administration followed by subsequent inoculations or other administrations. Thus, the composition may be administered in a single dose or in a plurality of doses. In one embodiment, the composition may be administered in multiple doses about months apart.

A composition may be administered alone or in combination with other treatments or standard vaccine, either simultaneously or sequentially dependent upon the condition to be treated. The composition can be administered after vaccination with and therefore act as a adjuvant for a vaccine. The composition may be contained in a capsule for chewing or may be contained in a gum for oral administration. The composition may be administered with a swab or may be administered with a nasal or oral spray. The composition may be contained in small particles suspended in the water or saline. The composition may also contain additional adjuvants, antibacterial agents or other pharmaceutically active agents as are conventional in the art. Adjuvants may include but are not limited to salts, emulsions (including oil/water compositions), saponins, liposomal formulations, virus particles, polypeptides, pathogen-associated molecular patterns (PAMPS), nucleic acid-based compounds or other formulations utilizing certain antigens. Suitable adjuvants include, e.g., vegetable oils, alum, Freund's incomplete adjuvant, or Freund's incomplete adjuvant, with oils and Freund's incomplete adjuvant being particularly preferred. Other adjuvants include agents such as aluminum hydroxide or phosphate (alum), immune-stimulating complexes (ISCOMs), synthetic polymers of sugars (CARBOPOL®), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. The composition may be contained in a mucosal bacterial toxin adjuvant such as the Escherichia coli labile toxi (LT) and cholera toxin (CT) or in CpG oligodeoxynucleotide (CpG ODN)^{[61]}. Other possible mucosal adjuvants include L3™ and Monophosphoryl lipid A (MPL), The vaccine may optionally include additional immune modulating substances such as cytokines or synthetic IFN-γ inducers such as poly I:C alone or in combination with the above-mentioned adjuvants. Still other adjuvants include microparticles or beads of biocompatible matrix materials. The microparticles may be composed of any biocompatible matrix materials as are conventional in the art, including but not limited to, agar and polyacrylates.

The practitioner skilled in the art will recognize that other carriers or adjuvants may be used as well. For example, Chitosan or any bioadhesive delivery system can be used, such as those described by Webb and Winkelstein^{[66]}.

The pharmaceutical composition containing S. pyogenes is preferably formulated for intranasal or intrapulmonary delivery using methods known in the art. The formulation of the S. pyogenes combined with the adjuvant is preferably selected to minimize side effects, such as inflammation, associated with vaccination or may improve the formulation's stability. The adjuvant may also have a role as an immunostimulant or as a depot. In some embodiments, the S. pyogenes composition is delivered by the refinement of a nebulizer or via three types of compact portable devices, the metered-dose inhaler (MDI) and the dry powder inhaler (DPI). Intranasal delivery can occur via the nasal spray, dropper or nasal metered drug delivery device. The inactive mycobacterium may be delivered via a metered dose inhaler. Typically, only 10-20% of the emitted dose is deposited in the lung. The high velocity and large particle size of the spray causes approximately 50-80% of the drug aerosol to impact in the oropharyngeal region. The composition may be contained in a dry powder formulation such as but not limited to a sugar carrier system. The Sugar Carrier System could include lactose surcrose, and/or glucose. Lactose and glucose are approved by the FDA as carriers. There are also larger sugar particles such as lactose monohydrate-typically 50-100 micrometers in diameter, which remain in the naso-oropharynx but allows the inactivated bacilli to travel through the respiratory tree into the alveoli.^{[102]} If desired, the composition may be contained in a liposomal formulation. Liposomes, like other inhaled particles reaching the alveoli, are cleared by macrophages. The processing, uptake and recycling of liposomal phospholipids occurs through the same mechanism as endogenous surfactant via the alveolar type II cells.

A pharmaceutical composition containing the irradiated mycobacterium described above is administered to a suitable individual for preventing or treating tuberculosis. The terms “individual,” “subject,” “host,” and “patient,” are used interchangeably herein and refer to any subject having a bacterial infection amenable to treatment using the therapeutic vaccine of the invention, and for whom treatment or therapy is desired. The pharmaceutical composition can be prepared for any mammalian host that is susceptible to infection by S. pyogenes. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom (c) preventing reinfection of the bacteria. Thus, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical or veterinarian.

The subject treated with the vaccine typically will have or will develop protective immunity to an infecting bacterium. The term “protective immunity” means that a vaccine, immunogenic composition or immunization schedule that is administered to a mammal induces an immune response that prevents, retards the development of, or reduces the severity of a disease that is caused by a pathogenic bacterium or diminishes or altogether eliminates the symptoms of the disease. By “infecting bacterium” is meant a bacterium that has established infection in the host, and which may be associated with a disease or undesirable symptom as a result. Generally, infecting bacteria are pathogenic bacteria.

The terms “immunogenic bacterial composition”, “immunogenic composition”, and “vaccine” are used interchangeably herein to mean a preparation capable of eliciting a cellular and/or humoral immune response in a subject when administered in a sufficient amount to elicit an immune response to epitopes present in said preparation.

### Composition Comprising Vitamin D Metabolites and Method of Using Same

In s further aspect, the invention relates to a composition of a form of Vitamin D and more particularly to a dose using metabolites, synthetic entities, and precursors of Vitamin D formulated for pulmonary and mucosal delivery.

Vitamin D occurs in many forms and is typically transformed by the liver to calcidiol. Subsequently, calcidiol is used to make calcitriol, a biologically active form of Vitamin D by either the kidneys or by monocytes/macrophages of the immune system. In the latter situation, the monocytes or macrophages produce calcitriol which acts locally as a cytokine against pathogens. The localized delivery of calcitriol to the mucosal and intrapulmonary surfaces of the lungs may provide applications as an adjuvant or therapeutic agent. Calcitriol has been shown to be a potent ligand of the vitamin D receptor and in vitro research has provided evidence for its use in the field of immunology.

The invention provides an adjuvant for a vaccine for preventing and/or treating bacterial borne disease. The composition containing of inactivated or attenuated bacteria and calcitriol can be utilized with a number of vaccination strategies: prophylactically, given prior to infection to prevent infection with bacteria and prophylactically, when it is administered post-exposure to eliminate or contain latent and prevent reactivation. It can also be used as a treatment for a bacterial, viral or fungal infection, pulmonary damage from toxins including cigarettes, any process causing interstitial lung disease or an autoimmune process. Finally, the composition can either be used to replace a current vaccine and/or as a booster to other vaccines in patients who have already the appropriate vaccination.

In one aspect, the invention provides a pharmaceutical composition comprising calcitriol, wherein the composition is formulated for intranasal, mucosal or intrapulmonary delivery to a mammalian host, and wherein the composition comprises an immunologically protective dose when delivered to the host.

In one aspect, the invention provides a pharmaceutical composition comprising calcitriol and one or more bacteria, wherein the composition is formulated for intranasal, mucosal or intrapulmonary delivery to a mammalian host, and wherein the composition comprises an immunologically protective dose when delivered to the host.

In one aspect, the invention provides a therapy for autoimmune diseases such as, rheumatoid arthritis, systemic lupus erythematosus, type I diabetes, encephalomyelitis, or inflammatory bowel disease.

Calcitriol may be used as a composition with irradiated or inactivated mycobacterial spercies (spp). Suitable Mycobacterium spp. include, e.g., M. tuberculosis, M. marinum, M. bovis, M. africanum, or M. microtti. In some embodiments, the inactivated Mycobacterium spp. cells are killed cells or cell lysates.

In general, any Mycobacterium species or strain that is a member of the M. tuberculosis complex can be used in the composition and methods of the invention. Suitable species, Mycobacterium which are members of the M tb complex include, e.g, Mycobacterium bovis, Mycobacterium africanum, Mycobacterium microtti, and Mycobacterium tuberculosis. Genetically-similar mycobacterium include Mycobacterium canettii and Mycobacterium marinum. The particular species or combination of species is selected for the corresponding host species and type Mycobacterium-associated disease to be treated. Other Mycobacteria that cause disease in humans include, e.g., Mycobacterium avium intracellulare, Mycobacterium leprae, Mycobacterium lepraemurium, Mycobacteria paratuberculosis, Mycobacterium ulcerans, Mycobacterium smegmatis, Mycobacterium xenopi, Mycobacterium chelonei, Mycobacterium fortuitum, Mycobacterium farcinogenes, Mycobacterium flavum, Mycobacterium haemophitum, Mycobacterium kansasii, Mycobacterium phlei, Mycobacterium scrofulaceum, Mycobacterium senegalense, Mycobacterium simiae, Mycobacterium thermoresistible, and Mycobacterium xenopi.

Additional suitable bacteria include, e.g., Acetobacter aurantius, Acinetobacter baumannii, Actinomyces israelii, Agrobacterium radiobacter, Agrobacterium tumefaciens, Azorhizobium caulinodans, Azotobacter vinelandii, Anaplasma, Anaplasma phagocytophilum, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaminogenicus, Bartonella, Bartonella henselae, Bartonella quintana, Bordetella, Bordetella bronchiseptica, Bordetella pertussis, Borrelia burgdorferi, Brucella, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila, Chlamydophila pneumoniae, Chlamydophila psittaci, Clostridium, Clostridium botulinum, Clostridium difficile, Clostridium perfringens, Clostridium tetani, Corynebacterium, Corynebacterium diphtheria, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenza, Haemophilus parainfluenzae, Haemophilus pertussis, Haemophilus vaginalis, Helicobacter pylori, Klebsiella pneumonia, Lactobacillus, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumonia, Lactobacillus Bulgaricus, Neisseria, Neisseria gonorrhoeae, Neisseria meningitides, Pasteurella, Pasteurella multocida, Pasteurella tularensis, Peptostreptococcus, Porphyromonas gingivalis, Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumonia, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholera, Vibrio comma, Vibrio parahaemolyticus, Vibrio vulnificus, Wolbachia, Yersinia, Yersinia enterocolitica, Yersinia pestis, Yersinia pseudotuberculosis.

In some embodiments, the cells are killed cells or cell lysates.

In some embodiments, some of the bacteria are inactivated or attenuated.

In some embodiments, the bacteria is inactivated with irradiation. Preferably irradiation is with gamma irradiation but other types of radiation may be used including xray and microwaves.

In other embodiments, the bacteria is inactivated with formalin or heat.

In some embodiments, the bacteria is inactivated with osmotic pressure via salts or drying process.

The pharmaceutical composition may optionally include an adjuvant to enhance an immune response in the host.

The pharmaceutical composition may optionally include a pharmaceutically acceptable carrier, or be provided lyophilized.

The pharmaceutical composition may optionally include a pharmaceutically acceptable carrier such as glucose, lactose or sorbitol.

In some embodiments, the pharmaceutical composition is formulated for intranasal delivery to the host.

In addition, the pharmaceutical composition is provided as an aerosol or spray package.

In one embodiment, the invention provides a pharmaceutical composition that includes a gamma-irradiated Mycobacterium spp. that is formulated for intranasal or intrapulmonary delivery to a mammalian host and which confers an immunologically protective dose when delivered to the host, e.g., a human.

In another aspect, the invention provides a method of vaccinating a mammal against TB. The method includes administering to the mammal a composition comprising inactivated Mycobacterium spp., wherein the vaccination of the mammal is intranasal or intrapulmonary, and wherein the composition comprises an immunologically protective dose when delivered to the host.

In another aspect, the invention provides an immunostimulant that facilitates delivery of another antigen.

In one aspect, the invention provides a pharmaceutical composition comprising a calcitriol and gamma irradiated Mycobacterium spp., wherein the composition is formulated for intranasal, mucosal or intrapulmonary delivery to a mammalian host, and wherein the composition comprises an immunologically protective dose when delivered to the host. In some embodiments, the inactivated Mycobacterium spp. cells are killed cells or cell lysates. When the subject is a human, 100% of the Mycobacterium spp. cells are preferably inactivated. In some embodiments, the Mycobacterium spp. for use in the method is inactivated with irradiation. Preferably irradiation is with gamma irradiation. In other embodiments, the Mycobacterium spp. is inactivated with formalin or heat.

The pharmaceutical composition for use in the method may optionally include an adjuvant to enhance a protective immune response in the host.

The pharmaceutical composition for use in the method may optionally include a pharmaceutically acceptable carrier, or be provided lyophilized.

In some embodiments, the pharmaceutical composition for use in the method is formulated for intranasal delivery to the host.

In addition, the pharmaceutical composition for use in the method is provided as an aerosol or spray package.

In some embodiments, the pharmaceutical composition is delivered through a device configured for nasal or pulmonary delivery.

In a still further aspect, the invention provides a method for preparing a vaccine for treating Mycobacterium infection, comprising formulating an immunologically protective dose of an inactivated Mycobacterium spp. for intranasal or pulmonary delivery to a mammalian host.

In some embodiments, the method includes testing the vaccine in a non-human animal model of tuberculosis. The animal model can be, e.g., a mouse, guinea pig, rabbit, bovine, or non-human primate.

A composition according to the invention is prepared using one or more forms of Vitamin D that is then formulated for delivery, preferably pulmonary and mucosal delivery to a subject. The Vitamin D composition, when delivered to the lung or mucosal/nasal mucosa of a subject is postulated to elicit an antimicrobial immune response and has been observed in vitro with monocytes in human blood.

The history of vitamin D has intrigued the medical community as far back as the 1800s when cod liver oil was used as a treatment for tuberculosis.^{[103]} It is now believed the healing benefits gained from sanatoriums was secondary to sunlight exposure and the body's innate production of Vitamin D. Vitamin D is not only obtained from ultraviolet light, but is found in oily fish, eggs and is fortified in some food products.

While Vitamin D itself is biologically inactive, it can be metabolized to biologically active forms. After Vitamin D is consumed within diet or produced by the epidermis, it circulates and is ultimately transported to the liver. The liver then hydroxylates the Vitamin D to form 25-hydroxyvitamin D (calcidiol or 25(OH)D3 or 25D3) which is the predominant form found in circulation. The kidney performs a second hydroxylation of 25-hydroxyvitmain D using an enzyme D3-1-hydroxylase enzyme to create 1,25-dihydroxyvitamin D (calcitriol, 1alpha,25-dihydroxyvitamin D or 1,25(OH)₂D3 or 1,25D3). Calcitriol is considered the most potent steroid hormone derived from cholecalciferol and is thought to be responsible for most of the effects within the body.

Calcitriol enters the nucleus of a cell, 1,25-dihydroxyvitamin D associates with the Vitamin D Receptor (VDR) and promotes its association with the retinoic acid X receptor (RXR).^{[104]} In the presence of 1,25-dihydroxyvitamin D the VDR/RXR complex initiates a cascade of molecular interactions that modulate the transcription of more than 50 genes in tissues throughout the body including in bone and intestine, mammary glands, colon, prostate, hematopoietic cells, and skin.^{[105]}

Vitamin D deficiency reduces the ability of macrophages to develop and to present macrophage-specific surface antigens. Moreover, the deficiency has been shown reduce the production of lysosomal enzyme acid phosphatase, and to use H2O2, a function integral to a macrophages antimicrobial function.^{[106][107]} Moreover, vitamin D, or lack of it, may be partially responsible for the seasonal increases of influenza during the wintertime.^{[108]}

It has been observed that populations with lower Vitamin D have increased incidence of tuberculosis. Strachan observed 8.5 fold increased risk of tuberculosis among vegetarian Hindu Asians immigrants in London from the Indian subcontinent compared to Muslims who ate meat and fish daily.^{[109]} Additionally, it has been shown that African-American individuals, known to have increased susceptibility to tuberculosis, had low 25-hydroxyvitamin D and were inefficient in supporting cathelicidin messenger RNA induction.^{[110][111][112]} Together, these insights support a link between toll like receptors and calcitriol mediated innate immunity in humans.

There is evidence to show that vitamin D suppress growth of M.tb within macrophages,^{[113][114,115]} and vitamin D stimulated toll like receptors that may provide innate immunity against M. tb.^{[116]} Crowle et al. confirmed that 1,25 D enabled macrophages to slow and stop bacillary replication even at extremely low concentrations. In fact, the protection against bacillary growth was achieved when 1,25D was at concentrations of 1,000 times lower than usual circulating levels and was induced even when 1,25D was added three days after infection. Here, Crowle used a concentration of 4 μg/ml which is high than normal circulating levels but offered concentrations within the granulomas. Thus this research provides evidence that 1,25 D as a immunomodulator and can help activate human macrophages to express immunity.^{[115]}

Early in 2001 Denis et al. showed that Calcitriol (1,25 (OH2), vitamin D3) alone, at doses up to 10⁹ M endowed human monocytes with a significant ability to restrict tuberculosis growth in vitro.^{[117]} A further analysis of in vitro mechanism of action by Liu^{[116]} showed that activation of human macrophages by a mycobacterial peptide induced expression of the VDR as well as Cyp27B1, a vitamin D-1-hydroxylase that converts inactive provitamin D [25(OH)D3] into the active 1,25(OH)₂D3. Moreover, exposure of macrophages with 1,25(OH)2D3 induces expression of the anti-microbial peptide cathelicidin and promotes killing of M.tb within the phagolysosome. Moreover, in monocytes infected with M. bovis Bacille Calmette Guerin, cathelicidin and 1,25(OH)2D3 were observed within the phagolysosome. The induction of TLR 2/1 reduced the survivability of intracellular M. tuberculosis in human monocytes and macrophages but not in monocytes-derived dendritic cells^{[116]} While the catheliciden pathway seems to be a result of evolution and can not be found in mice, the activation of the VDR in primary human monocytes triggers induction of at least one known antimicrobial peptide with antimicrobial properties as evidence from reduced colony forming units after the addition of 1,25(OH)2D3 to primary human macrophages infected with virulent M. tb.

Mucosal delivery of calcitriol in conjunction with attenuated or inactivated bacteria will aids in the engulfment and processing of the bacterium to allow for macrophage antigen presentation and imbue an enhanced immune response. The calcitriol stimulates catheliciden within the macrophage vacuoles to kill and disassemble the bacteria's antigenic components. Vitamin D has been linked with Toll-Like Receptor signaling and presentation of macrophages with vitamin D-1-hydroxylase may induce expression of an anti-microbial peptide cathelicidin to promote sufficient killing of M. tb.^{[118]} A further enhancement may be to use different metabolic states of mycobacterium added to a Vitamin D composition. This may have the potential of improving M. tb antigen presentation to the cellular immune response

One reason the inventors the use and formulation of calcitriol has been overlooked is that the proposed invention relies on the inventor's unique and proprietary insights with irradiated mycobacterium tuberculosis. Thus, the need was not apparent or readily assumed. The inventors proposed in an earlier invention to use aerosolize irradiated mycobacterium tuberculosis as a means to promote immunity and have unpublished data to support its use. As calcitriol may promote further antigen presentation of the macrophage, the inventors hypothesize that calcitriol when administered with irradiated mycobacterium will foster a boosted immune response. Moreover, the route of administration follows that calcitriol delivered to the mucosa may offer additional roles as an immunomodulator, therapeutic agent, and adjuvant.

The bacterium to be used in the pharmaceutical composition can include whole cells or portions of cells, e.g., cell lysates. For example, suitable components include a gamma irradiated whole cell lysate, gamma irradiated culture filtrate proteins, gamma irradiated cell wall fraction, gamma irradiated cell membrane fraction, gamma irradiated cytosol fraction, gamma irradiated soluble cell wall proteins, and gamma irradiated soluble protein pool.

1,25 D3 may have a role in the treatment and prevention of autoimmune diseases. The research of DeLuca et al. supports the idea that 1,25-dihydroxyvitamin D3 in the presence of a normal or high calcium diet can either prevent or markedly suppress in models autoimmune encephalomyelitis, rheumatoid arthritis, systemic lupus erythematosus, type I diabetes, and inflammatory bowel disease^{[119]}. DeLuco postulates that vitamin D stimulates transforming growth factor TGFb-1 and interleukin 4 (IL-4) production, which in turn may suppress inflammatory T cell activity. Moreover, it has been shown that polymorphisms of the vitamin d receptor have been responsible for increased risk for breast cancer.^{[120]} Low levels of vitamin D have been correlated with breast cancer disease progression and tissues associated with metastasized colon cancer fail to respond to calcitriol^{[121]} Thus, inventors believe that there is warranted evidence to test aerosolized vitamin d as a therapeutic or preventative agent in the cancer model.

The present invention additionally includes the use of different sugars as fine and coarse carriers as part of the composition for calcitriol delivery to respiratory and mucosal tissues. The aerosolized calcitriol may be used as part of a composition containing bacteria or viral components either as whole entities or as partial components. The localized delivery of calcitriol to mucosal and intrapulmonary surfaces of the lungs may provide applications as an adjuvant or therapeutic agent.

### Preparing Pharmaceutical Compositions

Calcitriol or Calcidiol is prepared for administration to a host by combining with a pharmaceutically acceptable carrier to form a pharmaceutical composition. Calcitriol and Calcidol are well known in the art.

The carrier can be glucose, sucrose, lactose, sorbitol, e.g., such as physiological saline, mineral oil, vegetable oils, aqueous sodium carboxymethyl cellulose, or aqueous polyvinylpyrrolidone.

Calcidiol may also be treated with the converting enzyme such as vitamin D-1-hydroxylase at time of therapy to convert to Calcitriol so as to prolong the half life of Calcitriol and avert storage problems. For example, the conversion may be executed in a device prior to administration.

Calcitriol is prepared for administration to a host by combining inactivated cells or cell lysates with a pharmaceutically acceptable carrier to form a pharmaceutical composition. The carrier can be glucose, sucrose, lactose, sorbitol, e.g., such as physiological saline, mineral oil, vegetable oils, aqueous sodium carboxymethyl cellulose, or aqueous polyvinylpyrrolidone. In some embodiments, the carrier is sufficiently pure to be administered therapeutically to a human subject. The artisan can readily prepare suitable solutions using, for example, isotonic vehicles such as Sodium Chloride Injection, Ringer's Injection, or Lactated Ringer's Injection. Preservatives, stabilizers, buffers, antioxidants and/or other additives may be included, as required.

A skilled person in the field familiar with the protocols, formulations, dosages and clinical practice associated with, e.g., the administration of calcitriol or calcidiol can in addition readily adapt these protocols for use with pharmaceutical compositions of the present invention. The immunomodulating form of Vitamin D is administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired.

Suitable dosage ranges are dependent on etiology, use, dose, and condition of the host. Suitable regimens for initial administration and booster shots are also variable but are typified by an initial administration followed by subsequent inoculations or other administrations. Thus, the composition may be administered in a single dose or in a plurality of doses. In one embodiment, the composition may be administered in two doses about 0-12 months apart.

The composition might also use synthetic derivatives of calcitriol as either a substitute or in addition. Synthetic derivatives include but are not limited to calcipotriol, calcipotriene, tacalcitol, dihydrotachysterol, and ergocalciferol.

Synthetic derivatives include the deltanoids which are usually a result of structural changes in the C,D-ring and the side chain regions.

Other synthetic derivatives include: 1,25-Dihydroxy-26,27-hexafluorocholecalciferol, 1,25-Dihydroxy-22E-ene-26,27-hexafluorocholecalciferol, 1,25-Dihydroxy-23-yne-cholecalciferol, 1,25-Dihydroxy-16-ene-23-yne-cholecalciferol, 1,25S-Dihydroxy-26-trifluoro-22E-ene-cholecalciferol, 1,25-Dihydroxy-16,23E-diene-cholecalciferol, 1,25-Dihydroxy-16-ene-cholecalciferol, 1,25-Dihydroxy-16-ene-23-yne-26,27-hexafluorocholecalciferol, 1,25-Dihydroxy-16,23Z-diene-26,27-hexafluorocholecalciferol, 1,25-Dihydroxy-16,23E-diene-26,27-hexafluorocholecalciferol, 1,25-Dihydroxy-16,23E-diene-26,27-hexafluoro-19 norcholecalciferol, 1,25-Dihydroxy-16-ene-yne-26,27-hexafluoro-14-nor-cholecalciferol, 1,25-Dihydroxy-16,23Z-diene-26,27-hexafluoro-19-nor-cholecalciferol.

A composition may be administered alone or in combination with other treatments or standard vaccine, either simultaneously or sequentially dependent upon the condition to be treated. The composition can be administered after vaccination with and therefore act as a adjuvant for a vaccine.

The composition may be contained in small particles suspended in the water or saline. The composition may also contain additional adjuvants, antibacterial agents or other pharmaceutically active agents as are conventional in the art. Adjuvants may include but are not limited to salts, emulsions (including oil/water compositions), saponins, liposomal formulations, virus particles, polypeptides, pathogen-associated molecular patterns (PAMPS), nucleic acid-based compounds or other formulations utilizing certain antigens. Suitable adjuvants include, e.g., vegetable oils, alum, Freund's incomplete adjuvant, or Freund's incomplete adjuvant, with oils and Freund's incomplete adjuvant being particularly preferred. Other adjuvants include agents such as aluminum hydroxide or phosphate (alum), immune-stimulating complexes (ISCOMs), synthetic polymers of sugars (CARBOPOL®), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be used.

The composition may optionally be contained in a mucosal bacterial toxin adjuvant such as the Escherichia coli labile toxi (LT) and cholera toxin (CT) or in CpG oligodeoxynucleotide (CpG ODN)^{[61]}. Another possible mucosal adjuvant Monophosphoryl lipid A (MPL), a derivative and less toxic form of LPS, when combined with liposomes was found to induce mucosal immunoprotective responses^{[62]}. One new adjuvant designed for nasal vaccination, Eurocine L3™, has been shown to induce long-lasting immunity against TB in experimental animal models after intranasal administration^{[63,64,65]}. The adjuvant technology consists of a non-toxic pharmaceutical formulation based on a combination of endogenous and pharmaceutically accepted lipids. The vaccine may optionally include additional immune modulating substances such as cytokines or synthetic IFN-γ inducers such as poly I:C alone or in combination with the above-mentioned adjuvants.

Still other adjuvants include microparticles or beads of biocompatible matrix materials. The microparticles may be composed of any biocompatible matrix materials as are conventional in the art, including but not limited to, agar and polyacrylates. The practitioner skilled in the art will recognize that other carriers or adjuvants may be used as well. For example, Chitosan or any bioadhesive delivery-system which may be used are described by Webb and Winkelstein the contents of which are incorporated by reference herein.^{[66]}

The pharmaceutical composition containing calcitriol is preferably formulated for intranasal or intrapulmonary delivery using methods known in the art. The formulation of the calcitriol combined with the adjuvant is preferably selected to minimize side effects, such as inflammation, associated with vaccination or may improve the formulation's stability. The adjuvant may also have a role as an immunostimulant or as a depot.

In some embodiments, the calcitriol composition are delivered by the refinement of a nebulizer or via three types of compact portable devices, the metered-dose inhaler (MDI) and the dry powder inhaler (DPI). Intranasal delivery can occur via the nasal spray, dropper or nasal metered drug delivery device. The inactive mycobacterium may be delivered via a metered dose inhaler. Typically, only 10-20% of the emitted dose is deposited in the lung. The high velocity and large particle size of the spray causes approximately 50-80% of the drug aerosol to impact in the oropharyngeal region.

The composition may be contained in a dry powder formulation such as but not limited to a sugar carrier system. The sugar carrier system can include, e.g., lactose, sucrose, and/or glucose. Lactose and glucose are approved by the FDA as carriers. There are also larger sugar particles such as lactose monohydrate-typically 50-100 micrometers in diameter, which remain in the naso-oropharynx but allows the inactivated bacilli to travel through the respiratory tree into the alveoli.^{[67]}

If desired, the composition may be contained in a liposomal formulation. Liposomes, like other inhaled particles reaching the alveoli, are cleared by macrophages. The processing, uptake and recycling of liposomal phospholipids occurs through the same mechanism as endogenous surfactant via the alveolar type II cells.

A pharmaceutical composition containing the irradiated mycobacterium described above is administered to a suitable individual for preventing or treating tuberculosis. Compositions can be made using methods disclosed in Lighter et al., US20100112007. Reference herein to “tuberculosis” includes reference to pulmonary and extra-pulmonary tuberculi. The terms “individual,” “subject,” “host,” and “patient,” are used interchangeably herein and refer to any subject having a bacterial infection amenable to treatment using the therapeutic vaccine of the invention, and for whom treatment or therapy is desired. The pharmaceutical composition can be prepared for any mammalian host that is susceptible to infection by mycobacterium. Suitable mammalian hosts include, e.g., farm animals such as swine and bovine.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom (c) preventing reactivation of the disease in TB, i.e. preventing the bacilli from transitioning from a dormant to growth phase. Thus, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical or veterinarian.

The subject treated with the vaccine typically will have or will develop protective immunity to an infecting bacterium. The term “protective immunity” means that a vaccine, immunogenic composition or immunization schedule that is administered to a mammal induces an immune response that prevents, retards the development of, or reduces the severity of a disease that is caused by a pathogenic bacterium or diminishes or altogether eliminates the symptoms of the disease. By “infecting bacterium” is meant a bacterium that has established infection in the host, and which may be associated with a disease or undesirable symptom as a result. Generally, infecting bacteria are pathogenic bacteria.

The terms “immunogenic bacterial composition”, “immunogenic composition”, and “vaccine” are used interchangeably herein to mean a preparation capable of eliciting a cellular and/or humoral immune response in a subject when administered in a sufficient amount to elicit an immune response to epitopes present in said preparation.

### Compositions Containing Vitamin D and Methods of Using Same

In a further aspect the invention relates to a composition of a form of irradiated bacterium species with epigallocatechin-3-gallate, retinoic acid and/or Vitamin D and their respective metabolites, synthetic entities, and precursors formulated for pulmonary and mucosal delivery.

The ability of intracellular bacteria to evade the host immune response is due to the bacterium's ability to arrest phagosome maturation via upregulation of tryptophan-aspartate containing coat protein (TACO). The interference of phagosome maturation provides the opportunity for the intracellular pathogen to replicate and allow for further virulence. The combination of Vitamin D and Retinoic Acid as well as epigallocatechin-3-gallate have been shown to downregulate TACO expression, promote phagosome maturation, and subsequently reduce virulence of the intracellular pathogen. Thus mucosal or subcutaneous preparations of either Vitamin D and Retinoic Acid or Epigallocatechin-3-gallate to irradiated bacterium could provide new therapeutic and vaccination formulations.

In one aspect, the invention provides an adjuvant for a vaccine for preventing and/or treating bacterial borne disease. The composition containing of inactivated or attenuated bacteria, retinoic acid and calcitriol can be utilized with a number of vaccination strategies: prophylactically, given prior to infection to prevent infection with bacteria and prophylactically, when it is administered post-exposure to eliminate or contain latent and prevent reactivation. It can also be used as a treatment for a bacterial, viral or fungal infection or an autoimmune process. Finally, the composition can either be used to replace a current vaccine and/or as a booster to other vaccines in patients who have already the appropriate vaccination.

In a further aspect, the invention provides a pharmaceutical composition comprising of Vitamin D, Retinoic Acid and/or epigallocatechin-3-gallate, wherein the composition is formulated for intranasal, mucosal or intrapulmonary delivery to a mammalian host, and wherein the composition comprises an immunologically protective dose when delivered to the host.

In one aspect, the invention provides a pharmaceutical composition comprising Vitamin D, Retinoic Acid and/or epigallocatechin-3-gallate with one or more bacteria, wherein the composition is formulated for intranasal, mucosal or intrapulmonary delivery to a mammalian host, and wherein the composition comprises an immunologically protective dose when delivered to the host.

Vitamin D, Retinoic Acid and/or epigallocatechin-3-gallate may be used as a composition with irradiated or inactivated mycobacterial spercies (spp). Suitable Mycobacterium spp. include, e.g., M. tuberculosis, M. marinum, M. bovis, M. africanum, or M. microtti. In some embodiments, the inactivated Mycobacterium spp. cells are killed cells or cell lysates.

In general, any Mycobacterium species or strain that is a member of the M. tuberculosis complex can be used in the composition and methods of the invention. Suitable species, Mycobacterium which are members of the M tb complex include, e.g, Mycobacterium bovis, Mycobacterium africanum, Mycobacterium microtti, and Mycobacterium tuberculosis. Genetically-similar mycobacterium include Mycobacterium canettii and Mycobacterium marinum. The particular species or combination of species is selected for the corresponding host species and type Mycobacterium-associated disease to be treated. Other Mycobacteria that cause disease in humans include, e.g., Mycobacterium avium intracellulare, Mycobacterium leprae, Mycobacterium lepraemurium, Mycobacteria paratuberculosis, Mycobacterium ulcerans, Mycobacterium smegmatis, Mycobacterium xenopi, Mycobacterium chelonei, Mycobacterium fortuitum, Mycobacterium farcinogenes, Mycobacterium flavum, Mycobacterium haemophitum, Mycobacterium kansasii, Mycobacterium phlei, Mycobacterium scrofulaceum, Mycobacterium senegalense, Mycobacterium simiae, Mycobacterium thermoresistible, and Mycobacterium xenopi.

Additional suitable bacteria include, e.g., Acetobacter aurantius, Acinetobacter baumannii, Actinomyces israelii, Agrobacterium radiobacter, Agrobacterium tumefaciens, Azorhizobium caulinodans, Azotobacter vinelandii, Anaplasma, Anaplasma phagocytophilum, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides, Bacteroides fragilis, Bacteroides gingivalis, Bacteroides melaminogenicus, Bartonella, Bartonella henselae, Bartonella quintana, Bordetella, Bordetella bronchiseptica, Bordetella pertussis, Borrelia burgdorferi, Brucella, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila, Chlamydophila pneumoniae, Chlamydophila psittaci, Clostridium, Clostridium botulinum, Clostridium difficile, Clostridium perfringens, Clostridium tetani, Corynebacterium, Corynebacterium diphtheria, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenza, Haemophilus parainfluenzae, Haemophilus pertussis, Haemophilus vaginalis, Helicobacter pylori, Klebsiella pneumonia, Lactobacillus, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumonia, Lactobacillus Bulgaricus, Neisseria, Neisseria gonorrhoeae, Neisseria meningitides, Pasteurella, Pasteurella multocida, Pasteurella tularensis, Peptostreptococcus, Porphyromonas gingivalis, Pseudomonas aeruginosa, Rhizobium radiobacter, Rickettsia, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumonia, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholera, Vibrio comma, Vibrio parahaemolyticus, Vibrio vulnificus, Wolbachia, Yersinia, Yersinia enterocolitica, Yersinia pestis, Yersinia pseudotuberculosis.

In some embodiments, the bacteria are killed cells or cell lysates.

In some embodiments, some of the bacteria are inactivated or attenuated.

In some embodiments, the bacteria are inactivated with irradiation. Preferably irradiation is with gamma irradiation but other types of radiation may be used including xray and microwaves.

In other embodiments, the bacteria are inactivated with formalin or heat.

In some embodiments, the bacteria are inactivated with osmotic pressure via salts or drying process.

The pharmaceutical composition may optionally include an adjuvant to enhance an immune response in the host.

The pharmaceutical composition may optionally include a pharmaceutically acceptable carrier, or be provided lyophilized.

The pharmaceutical composition may optionally include a pharmaceutically acceptable carrier such as glucose, lactose or sorbitol.

In some embodiments, the pharmaceutical composition is formulated for intranasal delivery to the host.

In addition, the pharmaceutical composition is provided as an aerosol or spray package.

In one embodiment, the invention provides a pharmaceutical composition that includes a gamma-irradiated Mycobacterium spp. that is formulated for intranasal or intrapulmonary delivery to a mammalian host and which confers an immunologically protective dose when delivered to the host, e.g., a human.

In another aspect, the invention provides a method of vaccinating a mammal against TB. The method includes administering to the mammal a composition comprising inactivated Mycobacterium spp., wherein the vaccination of the mammal is intranasal or intrapulmonary, and wherein the composition comprises an immunologically protective dose when delivered to the host.

In another aspect, the invention provides an immunostimulant that facilitates delivery of another antigen.

In one aspect, the invention provides a pharmaceutical composition comprising a retinoic acid and calcitriol and gamma irradiated Mycobacterium spp., wherein the composition is formulated for intranasal, mucosal or intrapulmonary delivery to a mammalian host, and wherein the composition comprises an immunologically protective dose when delivered to the host. In some embodiments, the inactivated Mycobacterium spp. cells are killed cells or cell lysates. When the subject is a human, 100% of the Mycobacterium spp. cells are preferably inactivated. In some embodiments, the Mycobacterium spp. for use in the method is inactivated with irradiation. Preferably irradiation is with gamma irradiation. In other embodiments, the Mycobacterium spp. is inactivated with formalin or heat.

The pharmaceutical composition for use in the method may optionally include an adjuvant to enhance a protective immune response in the host.

The pharmaceutical composition for use in the method may optionally include a pharmaceutically acceptable carrier, or be provided lyophilized.

In some embodiments, the pharmaceutical composition for use in the method is formulated for intranasal delivery to the host.

In addition, the pharmaceutical composition for use in the method is provided as an aerosol or spray package.

In some embodiments, the pharmaceutical composition is delivered through a device configured for nasal or pulmonary delivery.

In a still further aspect, the invention provides a method for preparing a vaccine for treating Mycobacterium infection, comprising formulating an immunologically protective dose of an inactivated Mycobacterium spp. for intranasal or pulmonary delivery to a mammalian host.

In some embodiments, the method includes testing the vaccine in a non-human animal model of tuberculosis. The animal model can be, e.g., a mouse, guinea pig, rabbit, bovine, or non-human primate.

Epigallocatechin-3-Gallate with Irradiated Bacteria

M. tuberculosis has co-evolved with humans and has evolved survival and replication mechanisms despite host immune responses. Evasion and subsequent mycobacterial persistence is due to the ability of M. tuberculosis to inhibit the phagosome—lysosome fusion due in part to upregulation of TACO.
A major component of green tea polyphenols, epigallocatechin-3-gallate has the inherent capacity to down-regulate TACO gene transcription within human macrophages through its ability to inhibit Sp1 transcription factor. EGCG has been shown to block this activity thus acting as a down regulator of Sp1-dependent genes by suppressing binding capacity^{[122][123]} EGCG down-regulates TACO gene transcription in a dose-dependent manner and in vitro data indicate substantially reduced mycobacterium survival within macrophages as assessed through flow cytometry and colony counts. Moreover, EGCG completely inhibits M. tuberculosis survival within macrophages when used prior to M. tb exposure in vitro.^{[124]} Used alone, EGCG may not provide substantial benefits in vivo. However, used with a proprietary composition of irradiated aerosolized mycobacterium, the inventor theorizes that the combination will allow for upregulated antigen presentation and provide an improvement to previous patent applications. As the promising survival data of aerosolized irradiated M.tb has not been published and is unexpected, the combination could yield promising and surprising results.
Retinoic Acid with Irradiated Bacteria

Retinoic acid is a metabolite of vitamin A (retinol) retinoic acid (RA) that stimulates macrophages. Since tuberculous bacilli prey upon alveolar macrophages, there might be potential to use retinoic acid to help treat or prevent tuberculosis. Research in rats, given 3 times weekly oral doses for 3 and 5 weeks post infection with Mycobacterium tuberculosis strain H37Rv, showed a significant difference in the severity of tuberculosis histopathology between control and RA-treated rats. Oral administration of RA decreased the number of colony-forming units (CFU) in both lung and spleen at 3 and 5 wk after H37Rv infection. Moreover, increased presentation of CD4-positive and CD8-positive T cells, natural killer cells, and CD163-positive macrophages increased in the infected lung tissues of orally treated rats. Since orally administered RA significantly inhibits the in vivo growth of M. tuberculosis and the development of tuberculosis, there might be potential to use mucosal, subcutaneous, or oral compositions in combination with aerosolized irradiated M.tb.^{[125]}

Vitamin D with Irradiated Bacteria

It is envisioned that mucosal delivery of calcitriol in conjunction with attenuated or inactivated bacteria will aid in the engulfment and processing of the bacterium to allow for macrophage antigen presentation and imbue an enhanced immune response. The calcitriol stimulates catheliciden within the macrophage vacuoles to kill and disassemble the bacteria's antigenic components. Vitamin D has been linked with Toll-Like Receptor signaling and presentation of macrophages with vitamin D-1-hydroxylase may induce expression of an anti-microbial peptide cathelicidin to promote sufficient killing of M.tb.^{[118]} A further enhancement may be to use different metabolic states of mycobacterium added to a Vitamin D composition. This may have the potential of improving M. tb antigen presentation to the cellular immune response.

The inventors proposed in an earlier invention to use aerosolize irradiated mycobacterium tuberculosis as a means to promote immunity and have unpublished data to support its use. As calcitriol may promote further antigen presentation of the macrophage, the inventors hypothesize that calcitriol, when administered with irradiated mycobacterium, will foster a boosted immune response. Moreover, the route of administration follows that calcitriol delivered to the mucosa may offer additional roles as an immunomodulator, therapeutic agent, and adjuvant.

The bacterium to be used in the pharmaceutical composition can include whole cells or portions of cells, e.g., cell lysates. For example, suitable components include a gamma irradiated whole cell lysate, gamma irradiated culture filtrate proteins, gamma irradiated cell wall fraction, gamma irradiated cell membrane fraction, gamma irradiated cytosol fraction, gamma irradiated soluble cell wall proteins, and gamma irradiated soluble protein pool.

Vitamin D and Retinoic Acid with Irradiated Bacteria

Vitamin D and retinoic acid appears to synergistically restrict invasion of macrophages by pathogenic mycobacteria via the TACO mechanism.^{[126]} The combination of Vitamin D and Retinoic Acid allow a greater number of cells experience maturation of then mycobacterial phagosome when compared to isolated treatment groups. Thus a mucosal or subcutaneous formulation of Vitamin D and retinoic acid may provide additional improvements with the composition of aerosolized irradiated mycobacterium.

Vitamin D, Retinoic Acid and Epigallocatechin-3-Gallate with Irradiated Bacteria

The present invention additionally includes the use of different sugars as fine and coarse carriers as part of the composition for Vitamin D, Retinoic Acid and/or epigallocatechin-3-gallate delivery to respiratory and mucosal tissues. The aerosolized composition may be used as part of a composition containing bacteria or viral components either as whole entities or as partial components. The localized delivery of calcitriol, retinoic acid or EGCG to mucosal and intrapulmonary surfaces of the lungs may provide applications as an adjuvant or therapeutic agent.

Vitamin D, Retinoic Acid and/or epigallocatechin-3-gallate is prepared for administration to a host by combining with a pharmaceutically acceptable carrier to form a pharmaceutical composition. Vitamin D, Retinoic Acid and/or epigallocatechin-3-gallate are well known in the art.

The carrier can be glucose, sucrose, lactose, sorbitol, e.g., such as physiological saline, mineral oil, vegetable oils, aqueous sodium carboxymethyl cellulose, or aqueous polyvinylpyrrolidone.

Calcidiol may also be treated with the converting enzyme such as vitamin D-1-hydroxylase at time of therapy to convert to Calcitriol so as to prolong the half life of Calcitriol and avert storage problems. For example, the conversion may be executed in a device prior to administration.

Vitamin D, Retinoic Acid and/or epigallocatechin-3-gallate is prepared for administration to a host by combining inactivated cells or cell lysates with a pharmaceutically acceptable carrier to form a pharmaceutical composition. The carrier can be glucose, sucrose, lactose, sorbitol, e.g., such as physiological saline, mineral oil, vegetable oils, aqueous sodium carboxymethyl cellulose, or aqueous polyvinylpyrrolidone. In some embodiments, the carrier is sufficiently pure to be administered therapeutically to a human subject. The artisan can readily prepare suitable solutions using, for example, isotonic vehicles such as Sodium Chloride Injection, Ringer's Injection, or Lactated Ringer's Injection. Preservatives, stabilizers, buffers, antioxidants and/or other additives may be included, as required.

A skilled person in the field familiar with the protocols, formulations, dosages and clinical practice associated with, e.g., the administration of Vitamin D, Retinoic Acid and/or epigallocatechin-3-gallate can in addition readily adapt these protocols for use with pharmaceutical compositions of the present invention. The immunomodulating form of Vitamin D is administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired.

Suitable dosage ranges are dependent on etiology, use, dose, and condition of the host. Suitable regimens for initial administration and booster shots are also variable but are typified by an initial administration followed by subsequent inoculations or other administrations. Thus, the composition may be administered in a single dose or in a plurality of doses. In one embodiment, the composition may be administered inup to four doses about 0-12 months apart.

The composition might also use synthetic derivatives of Vitamin D, Retinoic Acid and/or epigallocatechin-3-gallate as either a substitute or in addition. Synthetic derivatives include but are not limited to calcipotriol, calcipotriene, tacalcitol, dihydrotachysterol, and ergocalciferol.

Synthetic derivatives include the deltanoids which are usually a result of structural changes in the C,D-ring and the side chain regions.

Other synthetic derivatives include: 1,25-Dihydroxy-26,27-hexafluorocholecalciferol, 1,25-Dihydroxy-22E-ene-26,27-hexafluorocholecalciferol, 1,25-Dihydroxy-23-yne-cholecalciferol, 1,25-Dihydroxy-16-ene-23-yne-cholecalciferol, 1,25S-Dihydroxy-26-trifluoro-22E-ene-cholecalciferol, 1,25-Dihydroxy-16,23E-diene-cholecalciferol, 1,25-Dihydroxy-16-ene-cholecalciferol, 1,25-Dihydroxy-16-ene-23-yne-26,27-hexafluorocholecalciferol, 1,25-Dihydroxy-16,23Z-diene-26,27-hexafluorocholecalciferol, 1,25-Dihydroxy-16,23E-diene-26,27-hexafluorocholecalciferol, 1,25-Dihydroxy-16,23E-diene-26,27-hexafluoro-19 nor-cholecalciferol, 1,25-Dihydroxy-16-ene-yne-26,27-hexafluoro-14-nor-cholecalciferol, 1,25-Dihydroxy-16,23Z-diene-26,27-hexafluoro-19-nor-cholecalciferol.

A composition may be administered alone or in combination with other treatments or standard vaccine, either simultaneously or sequentially dependent upon the condition to be treated. The composition can be administered after vaccination with and therefore act as a adjuvant for a vaccine.

The composition may be contained in small particles suspended in the water or saline. The composition may also contain additional adjuvants, antibacterial agents or other pharmaceutically active agents as are conventional in the art. Adjuvants may include but are not limited to salts, emulsions (including oil/water compositions), saponins, liposomal formulations, virus particles, polypeptides, pathogen-associated molecular patterns (PAMPS), nucleic acid-based compounds or other formulations utilizing certain antigens. Suitable adjuvants include, e.g., vegetable oils, alum, Freund's incomplete adjuvant, or Freund's incomplete adjuvant, with oils and Freund's incomplete adjuvant being particularly preferred. Other adjuvants include agents such as aluminum hydroxide or phosphate (alum), immune-stimulating complexes (ISCOMs), synthetic polymers of sugars (CARBOPOL®), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be used.

The composition may optionally be contained in a mucosal bacterial toxin adjuvant such as the Escherichia coli labile toxi (LT) and cholera toxin (CT) or in CpG oligodeoxynucleotide (CpG ODN)^{[61]}. Another possible mucosal adjuvant Monophosphoryl lipid A (MPL), a derivative and less toxic form of LPS, when combined with liposomes was found to induce mucosal immunoprotective responses^{[62]}. One new adjuvant designed for nasal vaccination, Eurocine L3™, has been shown to induce long-lasting immunity against TB in experimental animal models after intranasal administration^{[63,64,65]}. The adjuvant technology consists of a non-toxic pharmaceutical formulation based on a combination of endogenous and pharmaceutically accepted lipids. The vaccine may optionally include additional immune modulating substances such as cytokines or synthetic IFN-γ inducers such as poly I:C alone or in combination with the above-mentioned adjuvants.

Still other adjuvants include microparticles or beads of biocompatible matrix materials. The microparticles may be composed of any biocompatible matrix materials as are conventional in the art, including but not limited to, agar and polyacrylates. The practitioner skilled in the art will recognize that other carriers or adjuvants may be used as well. For example, Chitosan or any bioadhesive delivery-system which may be used are described by Webb and Winkelstein the contents of which are incorporated by reference herein.^{[66]}

The pharmaceutical composition containing epigallocatechin-3-gallate, retinoic acid and/or Vitamin D is preferably formulated for intranasal or intrapulmonary delivery using methods known in the art. The formulation of the calcitriol combined with the adjuvant is preferably selected to minimize side effects, such as inflammation, associated with vaccination or may improve the formulation's stability. The adjuvant may also have a role as an immunostimulant or as a depot.

In some embodiments, the Vitamin D, Retinoic Acid and/or epigallocatechin-3-gallate composition are delivered by the refinement of a nebulizer or via three types of compact portable devices, the metered-dose inhaler (MDI) and the dry powder inhaler (DPI). Intranasal delivery can occur via the nasal spray, dropper or nasal metered drug delivery device. The inactive mycobacterium may be delivered via a metered dose inhaler. Typically, only 10-20% of the emitted dose is deposited in the lung. The high velocity and large particle size of the spray causes approximately 50-80% of the drug aerosol to impact in the oropharyngeal region.

The composition may be contained in a dry powder formulation such as but not limited to a sugar carrier system. The sugar carrier system can include, e.g., lactose, sucrose, and/or glucose. Lactose and glucose are approved by the FDA as carriers. There are also larger sugar particles such as lactose monohydrate-typically 50-100 micrometers in diameter, which remain in the naso-oropharynx but allows the inactivated bacilli to travel through the respiratory tree into the alveoli.^{[67]}

If desired, the composition may be contained in a liposomal formulation. Liposomes, like other inhaled particles reaching the alveoli, are cleared by macrophages. The processing, uptake and recycling of liposomal phospholipids occurs through the same mechanism as endogenous surfactant via the alveolar type II cells.

A pharmaceutical composition containing the irradiated mycobacterium described above is administered to a suitable individual for preventing or treating tuberculosis. Compositions can be made using methods disclosed in Lighter et al., US20100112007. Reference herein to “tuberculosis” includes reference to pulmonary and extra-pulmonary tuberculi. The terms “individual,” “subject,” “host,” and “patient,” are used interchangeably herein and refer to any subject having a bacterial infection amenable to treatment using the therapeutic vaccine of the invention, and for whom treatment or therapy is desired. The pharmaceutical composition can be prepared for any mammalian host that is susceptible to infection by mycobacterium. Suitable mammalian hosts include, e.g., farm animals such as swine and bovine.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a mammalian subject, more particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom (c) preventing reactivation of the disease in latent TB, i.e. preventing the bacilli from transitioning from a dormant to growth phase. Thus, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical or veterinarian.

The subject treated with the vaccine typically will have or will develop protective immunity to an infecting bacterium. The term “protective immunity” means that a vaccine, immunogenic composition or immunization schedule that is administered to a mammal induces an immune response that prevents, retards the development of, or reduces the severity of a disease that is caused by a pathogenic bacterium or diminishes or altogether eliminates the symptoms of the disease. By “infecting bacterium” is meant a bacterium that has established infection in the host, and which may be associated with a disease or undesirable symptom as a result. Generally, infecting bacteria are pathogenic bacteria.

The terms “immunogenic bacterial composition”, “immunogenic composition”, and “vaccine” are used interchangeably herein to mean a preparation capable of eliciting a cellular and/or humoral immune response in a subject when administered in a sufficient amount to elicit an immune response to epitopes present in the preparation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

1. A pharmaceutical composition comprising an irradiated microbial spp., wherein said microbial spp. includes cells in a predefined metabolic state, wherein said predefined metabolic state is triggered by nutritional deprivation, iron availability, aerobic growth, anaerobic growth, microaerophilic, oxidative stress, exposure to carbon monoxide, altered pH, availability of nutrients, manipulated cell population density, antibiotic presence or a combination of two or more of these states.

2. (canceled)

3. The pharmaceutical composition of claim 1, wherein said Mycobacterium spp. is inactivated with irradiation such as gamma irradiation.

4. The pharmaceutical composition of claim 1, wherein a composition containing irradiated bacterium or its cell lysates wherein administration is parenteral, intravenous, subcutaneous, intradermal or intramuscular as part of a vaccination or treatment regimen.

5. The pharmaceutical composition of claim 1, wherein more than 90% of the mycobacterium cells are in the predetermined state.

6. The pharmaceutical composition of claim 1, wherein said composition is aerosolized and formulated for intranasal, mucosal or intrapulmonary delivery to a mammalian host whereby particle size for deep lung penetration is less than 7 microns.

7. The pharmaceutical composition of claim 1, further comprising an adjuvant, toll like receptor or pattern recognition receptor agonist, aluminum salt, or saponins.

8. The pharmaceutical composition of claim 1, further comprising a therapeutically effective amount of Bacille Calmette-Guerin (BCG).

9. A method of enhancing an immune response in a subject, the method comprising administering a pharmaceutically effective amount of the composition of claim 1.

10. The method of claim 9, wherein the microbial spp. is a Mycobacterium administered at a dose from 10² to 10¹² Mycobacterium spp.

11. The method of claim 9, wherein inactivated Mycobacterium spp includes mycobacterium cells or cell lysates.

12. The pharmaceutical composition of claim 1, comprising an irradiated Brucella microbial species, wherein said composition is formulated for vaginal, rectal, or gastrointestinal mucosal delivery with an osmotic delivery system or compositional matrix to a mammalian host, and wherein said composition comprises an immunologically stimulating dose when delivered to said host.

13. The pharmaceutical composition of claim 12 compromising a pharmaceutically acceptable carrier for gastrointestinal delivery.

14. The pharmaceutical composition of claim 12 wherein said inactivated spp. cells are provided as part of a feed regimen or delivered in conjunction with specialized plant based vaccines or seed crops such as rice, maize, or soybeans.

15. The pharmaceutical composition of claim 12 wherein said inactivated spp. cells are provided in a form suitable for gastric delivery with the use of pH-sensitive polymers that enhance gastric release, mucoadhesive polymers for gastric retention and release, or gastric retention systems.

16. The pharmaceutical composition of claim 12, wherein said inactivated spp. cells are provided in a form suitable for enteric delivery with pH-sensitive polymers that resist gastric dissolution, swelling/gelling HG for controlled release, or as a osmotic pressure-driven tablet or device.

17. The pharmaceutical composition of claim 12, wherein said inactivated spp. cells are provided in a form suitable for colonic delivery.

18. The pharmaceutical composition of claim 12, further comprising compositions degradable by colonic bacteria.

19. The pharmaceutical composition of claim 12, wherein colonic bacteria comprise one or more active azoreductase, esterase, amidase, glucosidase, or glucuronidase.

20. The pharmaceutical composition of claim 12, wherein said inactivated spp. cells are provided in form suitable for colonic delivery with osmotic or swelling systems that release at times well beyond gastric and enteric transit times.

21. The pharmaceutical composition of claim 12, wherein said inactivated spp. cells are coated with suitable polymers that degrade preferentially in the colon.

22. The pharmaceutical composition of claim 12, wherein said composition is coated with a pH-sensitive polymer.

23. The pharmaceutical composition of claim 22, wherein said pH-sensitive polymer is Eudragit L 100, Eudragit S 100, Eudragit L 30 D, Eudragit FS 30 D, Eudragit L 100-55, Polyvinyl acetate phthalate, Hydroxypropyl ethylcellulose phthalate, Hydroxypropyl ethylcellulose phthalate 50, Hydroxypropyl ethylcellulose phthalate 55, Cellulose acetate trimelliate or Cellulose acetate phthalate.

24. The pharmaceutical composition of claim 12 wherein said osmotic delivery is with a Rose Nelson pump, Higuchi Leeper pump, Higuchi Theeuwes pump, elementary osmotic pump, multichamber osmotic pump, OROS-CT, Multi particulate delayed release systems, Liquid Oral Osmotic System, Sandwiched osmotic tablet, Monolithic osmotic system, Osmotic bursting osmotic pump, or a telescopic capsule for delayed release.

25. The pharmaceutical composition of claim 12, further comprising a therapeutically effective amount of Bacille Calmette Guerin.

26. The method of claim 9, further comprising testing said composition for efficaciousness in treating or preventing Crohn's disease.

27. The method of claim 9, further comprising testing said composition for efficaciousness in treating or preventing Brucellosis disease.

28. The method of claim 9 further comprising testing said composition for treating or preventing Johne's disease.

29. The pharmaceutical composition of claim 1, comprising an immunologically effective dose of irradiated S. pyogenes formulated for intranasal, mucosal or intrapulmonary delivery to a mammalian host.

30. The pharmaceutical composition of claim 29, comprising one or more serotypes of S. pyogenes.

31. A method of treating or preventing a Streptococcus infection, the method comprising administering to a subject in need thereof an effective dose of the composition of claim 29.

32. The method of claim 31, wherein said composition is administered with inactivated mycobacterium spp. or another adjuvant.

33. The pharmaceutical composition of claim 1, further comprising Vitamin D, Retinoic Acid, or epigallocatechin-3-gallate, a noninfectious microbe, or a microbial particle wherein the said composition is formulated for intranasal, vaginal, rectal, mucosal or intrapulmonary delivery to a mammalian host, and wherein said composition comprises an immunologically stimulating dose when delivered to said host.

34. The pharmaceutical composition of claim 33 comprising calcidiol and the enzyme 25-hydroxyvitamin D3 1-alpha-hydroxylase, Retinoic Acid and/or epigallocatechin-3-gallate.

35. The pharmaceutical composition of claim 33, wherein said Vitamin D, Retinoic Acid and/or epigallocatechin-3-gallate is administered with glucose, sorbitol or lactose.

36. The pharmaceutical composition of claim 33, further comprising a synthetic form of Vitamin D, Retinoic Acid and/or epigallocatechin-3-gallate.

37. The pharmaceutical composition of claim 1, further comprising a pharmaceutically acceptable carrier and formulated for mucosal delivery.

38. The pharmaceutical composition of claim 1, wherein said composition is lyophilized.

39. An aerosol or spray package comprising the pharmaceutical composition of claim 1 configured for nasal or pulmonary delivery.

40. The pharmaceutical composition of claim 29, further comprising glucose, sorbitol or lactose.

41. The method of claim 9, wherein said composition is administered prophylactically or therapeutically to an animal model.

42. The method of claim 37, wherein said animal model is a mouse, guinea pig, rabbit, sheep, goat, bovine, non-human primate or human.

43. A method of using the composition of claim 1 as an adjuvant or immunostimulator, the method comprising administering to a mammalian subject without apparent Mycobacterium infection a dose of aerosolized Mycobacterium spp. sufficient to enhance an immune response in said subject to an antigen.

44. A method of treating or preventing asthma in an animal subject comprising administering to a subject in need thereof the composition of claim 1.

45. A method of treating or preventing cancer in a subject comprising administering to a subject in need thereof an effective amount of the composition of claim 1.

46. The pharmaceutical composition of claim 1, wherein the microbial spp. is aerosolized.
